(19) United States
(12) Patent Application Publication
(10) Pub. No.: US 2020/0181566 A1
(43) Pub. Date: Jun. 11, 2020

(54) MICROFLUIDIC THREE-DIMENSIONAL CELL CULTURE DEVICE

(71) Applicant: THE REGENTS OF THE UNIVERSITY OF MICHIGAN, OFFICE OF TECHNOLOGY TRANSFER, Ann Arbor, MI (US)

(72) Inventors: Koh Meng AW YONG, Ann Arbor, MI (US); Jianping FU, Ann Arbor, MI (US)

(21) Appl. No.: 16/789,231

(22) Filed: Feb. 12, 2020

Related U.S. Application Data
(63) Continuation of application No. PCT/US2018/046968, filed on Aug. 17, 2018.

(51) Int. Cl. C12N 5/00; C12M 1/12; C12M 3/06; C12M 1/00

(52) U.S. Cl. CPC C12N 5/0062; C12M 25/14; C12N 2513/00; C12M 31/10; C12M 23/16

(57) ABSTRACT

Described herein are various embodiments directed to microfluidic cell culture devices, systems, and methods. Embodiments of devices and systems disclosed herein may be used to grow and characterize one or more phenotypes of a cell sample. An apparatus may include an apparatus including a substrate defining a cavity, and further include a scaffold disposed within the cavity. The substrate and the scaffold may collectively define a set of channels including a first channel and a second channel. The first channel may be configured to receive and culture a cell sample during use. The second channel may be configured to receive a fluid during use. The scaffold may be configured to permit diffusion of the fluid through the scaffold and into the first channel.

### CROSS-REFERENCE TO RELATED APPLICATIONS

This application is a continuation of International Patent Application No. PCT/US2018/046968, filed on Aug. 17, 2018, which claims priority to U.S. Provisional Application Ser. No. 62/547,366, filed on Aug. 18, 2017, the disclosure of each of which is hereby incorporated by reference in their entirety.

### BACKGROUND

Analysis of invading leader cells at a tumor invasion front may be used as a diagnostic tool for disease and personalized medicine. Conventional two-dimensional (2D) tissue culture approaches may be unable to fully recapitulate the behavior of cells in their native three-dimensional (3D) environment. Patient derived xenograft (PDX) techniques may require specialized equipment and trained personnel to generate intra-vital imaging in animal models. Conventional microfluidic devices may be unable to recapitulate the in situ histology seen in organoid and/or animal models due to their small size and may be unsuitable for long-term cell culture. Therefore, additional devices, systems, and methods for culturing cells may be desirable.

### SUMMARY

In general, a method includes applying a cell sample to a first channel of an apparatus, the apparatus including a substrate defining a cavity and further define a scaffold disposed within the cavity. The substrate and the scaffold may collectively define a set of channels including the first channel and a second channel parallel to the first channel. External fluid flow may be prohibited through the first channel after applying the cell sample to the first channel. Fluid may flow through the second channel using a fluid pump for at least about 21 days such that the first channel is subject to indirect interstitial pressure from the fluid flowing through the second channel.

In some embodiments, signal data may be received corresponding to the cell sample at a set of predetermined time intervals. A cell sample data may be generated from the detector. One or more phenotypes of the cell sample may be identified from the cell sample data. Each of the phenotypes of the one or more phenotypes may be selected from the group consisting of cell morphology, migration speed, DNA, RNA, and protein. The fluid flow may include flowing the fluid through the second channel in a closed loop path. The first channel and the second channel may have a center-to-center distance of between about 1 mm and about 5 mm. A set of anchors may be attached to an interface between the substrate and the scaffold. In some of these embodiments, the set of anchors includes attached a first anchor to the first channel and attaching a second anchor to the second channel. In some of these embodiments, a length of a portion of the first anchor disposed wholly in the first channel is greater than a length of a portion of the second anchor disposed wholly in the second channel. In some embodiments, the scaffold is enclosed and sealed from an external environment. In some embodiments, formation of air bubbles may be prohibited through the second channel by holding the air bubbles in a fluid source upstream of the second channel. In some embodiments, a set of vents may be formed in the scaffold. The set of vents may be coupled to the first channel to allow air to escape when the cell sample is applied to the first channel.

In some of these embodiments, the set of channels may include a third channel having a center-to-center distance from the first channel of between about 1 mm and about 5 mm. In some of these embodiments, each channel of the set of channels is on a same plane. In some of these embodiments, at least one channel of the set of channels is on a different plane than the other channels of the set of channels. In some of these embodiments, fluid may flow through the third channel for a set of predetermined time periods. In some of these embodiments, the fluid flowing in the second channel is a first fluid, and the fluid flowing in the third channel is a second fluid different from the first fluid. In some embodiments, external fluid flow may be prohibited through the third channel after applying the cell sample to the first channel. In some of these embodiments, at least one of the first fluid and the second fluid is a growth medium. In some embodiments, flowing fluid through the second channel and the third channel is in a same direction. In some embodiments, flowing fluid through the second channel and the third channel is in an opposite direction.

In some of these embodiments, the cell sample has a volume of at least about 20 μL. In some embodiments, one or more of phosphate buffered saline and fetal bovine serum may be applied to the first channel. The cell sample may include one or more of cancer cells including prostate cancer cells. The cell sample may include one or more mammalian cells including cancer cells, epithelial cells, fibroblasts, immune cells, endothelial cells, and nerve cells. Each channel of the set of channels may have a diameter of between about 0.2 mm and about 2.0 mm. The first channel may be configured to receive between about 10⁷ cells and about 10⁵ cells. The substrate may be composed of one or more of polysiloxane, polydimethylsiloxane, polystyrene, polyethylene, and polycarbonate. The scaffold may be an extracellular matrix. The extracellular matrix may be composed of one or more of collagen, hydrogel, agarose, polyethylene glycol, alginate, hyaluronan acid, laminin, fibronectin, proteoglycans, and elastin. The collagen may include a concentration between about 1 mg/ml and about 10 mg/ml. The substrate may be composed of polydimethylsiloxane in a 11:1 polymer-to-cross-linker volume ratio. The substrate may be composed of polydimethylsiloxane in a polymer-to-cross-linker volume ratio between about 10:1 to about 30:1. The fluid flowing through the second channel may flow at a rate of between about 1 μL/min and about 200 μL/min. The fluid flowing through the third channel may flow at a rate of between about 1 μL/min and about 200 μL/min. The set of predetermined time periods may collectively be between about 21 days and about 60 days. The fluid may flow continuously. A portion of the scaffold between the first channel and the second channel may have a pressure of between about 0 Pa and about 200 Pa during the fluid flow. A portion of the scaffold between the first channel and the third channel may have a pressure of between about 0 Pa and about 200 Pa during the fluid flow.

In some embodiments, the cell sample may have a first cell sample. The apparatus is a first apparatus, the substrate is a first substrate, the cavity is a first cavity, the scaffold is a first scaffold, and the set of channels is a first set of channels. A second cell sample may be applied to a first channel of a second apparatus. The second apparatus may include a second substrate defining a second cavity and a second scaffold disposed within the second cavity. The second substrate and the second scaffold may collectively define a second set of channels including the first channel and a second channel parallel to the first channel. External fluid flow may be prohibited through the second channel after applying the cell sample to the first channel. The fluid may flow through the second channel using the fluid pump for at least about 21 days such that the first channel is subject to indirect interstitial pressure from the flowing fluid through the second channel. The cell sample may be a first cell sample. Signal data may be received corresponding to the second cell sample at a set of predetermined time intervals. Second cell sample data may be generated from the detector. One or more phenotypes of the second cell sample may be identified from the second cell sample data.

In some embodiments, a method may include applying a cell sample to a first channel of an apparatus, the apparatus including a substrate defining a cavity and an extracellular matrix disposed within the cavity. The substrate and the extracellular matrix may collectively define a set of channels including a first channel configured to three-dimensionally culture a cell sample and a second channel parallel to the first channel. Fluid may flow through the second channel using a fluid pump for at least about 21 days.

In some embodiments, external fluid flow may be prohibited through the first channel after applying the cell sample to the first channel such that the first channel is subject to indirect interstitial pressure from the flowing fluid through the second channel. A portion of the cell sample may be separated from the extracellular matrix. The cell sample may be enzymatically dissociated into an invasive portion and a non-invasive portion. One or more phenotypes of one or more of the invasive portion and the non-invasive portion may be separately identified.

In some embodiments, the phenotypes may include one or more of cell morphology, migration speed, DNA, RNA, and protein. The fluid may flow through the second channel in a closed loop path. The first channel and the second channel may have a center-to-center distance of between about 1 mm and about 5 mm. A set of anchors may be attached to an interface between the substrate and the extracellular matrix. The set of anchors includes a first anchor attached to the first channel and a second anchor attached to the second channel. A length of a portion of the first anchor disposed wholly in the first channel is greater than a length of a portion of the second anchor disposed wholly in the second channel. The substrate may be enclosed and sealed from an external environment. Formation of air bubbles may be prohibited through the second channel by holding the air bubbles in a fluid source upstream of the second channel.

In some embodiments, a set of vents may be formed in the scaffold. The set of vents may be coupled to the first channel to allow air to escape when the cell sample is applied to the first channel. In some embodiments, the set of channels may include a third channel having a center-to-center distance from the first channel of between about 1 mm and about 5 mm. Each channel of the set of channels is on a same plane. At least one channel of the set of channels is on a different plane than the other channels of the set of channels. Fluid may flow through the third channel for a set of predetermined time periods. In some embodiments, the fluid flowing in the second channel is a first fluid, and the fluid flowing in the third channel is a second fluid different from the first fluid. External fluid flow may be prohibited through the third channel after applying the cell sample to the first channel.

In some embodiments, at least one of the first fluid and the second fluid is a growth medium. In some embodiments, flowing fluid through the second channel and the third channel may be in a same direction. In some embodiments, flowing fluid through the second channel and the third channel may be in an opposite direction. In some embodiments, the cell sample may have a volume of at least about 20 μL. In some embodiments, one or more of phosphate buffered saline and fetal bovine serum may be applied to the first channel.

In some embodiments, the cell sample may include one or more of cancer cells including prostate cancer cells. The cell sample may include one or more mammalian cells including cancer cells, epithelial cells, fibroblasts, immune cells, endothelial cells, and nerve cells. Each channel of the set of channels may have a diameter of between about 0.2 mm and about 2.0 mm. The first channel may be configured to receive between about 10⁷ cells and about 10⁵ cells. The substrate may be composed of one or more of polysiloxane, polydimethylsiloxane, polystyrene, polyethylene, and polycarbonate.

In some embodiments, the scaffold may be an extracellular matrix. The extracellular matrix may be composed of one or more of collagen, hydrogel, agarose, polyethylene glycol, alginate, hyaluronan acid, laminin, fibronectin, proteoglycans, and elastin. In some embodiments, the collagen may include a concentration between about 1 mg/ml and about 10 mg/ml. The substrate may be composed of polydimethylsiloxane in a 11:1 polymer-to-cross-linker volume ratio. The substrate may be composed of polydimethylsiloxane in a polymer-to-cross-linker volume ratio between about 10:1 to about 30:1. In some embodiments, the fluid flowing through the second channel flows at a rate of between about 1 μL/min and about 200 μL/min. In some embodiments, the fluid flowing through the third channel flows at a rate of between about 1 μL/min and about 200 μL/min. In some embodiments, the set of predetermined time periods may collectively be between about 21 days and about 60 days. In some embodiments, the fluid may flow continuously.

In some embodiments, a portion of the scaffold between the first channel and the second channel may have a pressure of between about 0 Pa and about 200 Pa during the fluid flow. In some embodiments, a portion of the scaffold between the first channel and the third channel may have a pressure of between about 0 Pa and about 200 Pa during the fluid flow. In some embodiments, the cell sample may be a first cell sample, the apparatus is a first apparatus, the substrate is a first substrate, the cavity is a first cavity, the scaffold is a first scaffold, and the set of channels is a first set of channels. A second cell sample may be applied to a first channel of a second apparatus, the second apparatus including a second substrate defining a second cavity and a second scaffold disposed within the second cavity. The second substrate and the second scaffold may collectively define a second set of channels including the first channel and a second channel parallel to the first channel. External fluid flow may be prohibited through the second channel after applying the cell sample to the first channel. In some embodiments, the fluid may flow through the second channel using the fluid pump for at least about 21 days such that the first channel is subject to indirect interstitial pressure from the flowing fluid through the second channel. The cell sample may be a first cell sample. Signal data may be received corresponding to the second cell sample at a set of predetermined time intervals. Second cell sample data may be generated from the detector. One or more phenotypes of the second cell sample may be identified from the second cell sample data.

In some embodiments, a method of manufacturing an apparatus may include forming a substrate defining a cavity. An extracellular matrix may be formed within the cavity. The substrate and the extracellular matrix may collectively define a set of channels including a first channel configured to three-dimensionally culture a cell sample and a second channel parallel to the first channel. A substantially transparent layer may be coupled to the substrate to enclose and seal the extracellular matrix from an external environment.

In some embodiments, forming the substrate may include polymerizing polydimethyl siloxane disposed over a set of parallel rods. The rods may be removed from the substrate such that the substrate defines a portion of the set of channels. Forming the cavity in the substrate. In some embodiments, forming the extracellular matrix may include inserting the set of parallel rods through the extracellular matrix. The extracellular matrix disposed over the set of parallel rods may be polymerized within the cavity. The set of parallel rods may be removed from the substrate and the extracellular matrix such that the substrate and the extracellular matrix defines the set of channels. The substrate and the substantially transparent layer may be formed using one or more of die cutting, extrusion, additive manufacturing, stereolithography, fused deposit modeling, and injection molding.

In some embodiments, the substrate and the substantially transparent layer are coupled using one or more of adhesives, ultrasonic welding, laser welding, and solvent bonding. The first channel and the second channel have a center-to-center distance of between about 1 mm and about 5 mm. A set of anchors may be attached to an interface between the substrate and the extracellular matrix. In some embodiments, attaching the set of anchors may include a first anchor attached to the first channel and a second anchor attached to the second channel. In some embodiments, the set of channels includes a third channel having a center-to-center distance from the first channel of between about 1 mm and about 5 mm. In some embodiments, each channel of the set of channels is on a same plane. At least one channel of the set of channels is on a different plane than the other channels of the set of channels.

In some embodiments, the cell sample may have a volume of at least about 20 μL. In some embodiments, one or more of phosphate buffered saline and fetal bovine serum may be applied to the first channel. In some embodiments, the cell sample may include one or more of cancer cells including prostate cancer cells. In some embodiments, each channel of the set of channels has a diameter of between about 0.2 mm and about 2.0 mm. In some embodiments, the first channel may be configured to receive between about 10⁷ cells and about 10⁵ cells. In some embodiments, the substrate may be composed of one or more of polysiloxane, polydimethylsiloxane, polystyrene, polyethylene, and polycarbonate. The extracellular matrix may be composed of one or more of collagen, hydrogel, agarose, polyethylene glycol, alginate, hyaluronan acid, laminin, and fibronectin. The collagen may include a concentration between about 1 mg/ml and about 2.5 mg/ml. The substrate may be composed of polydimethylsiloxane in a 11:1 polymer-to-cross-linker volume ratio. In some embodiments, a set of vents may be formed in the scaffold. The set of vents may be coupled to the first channel to allow air to escape when the cell sample is applied to the first channel.

In some embodiments, a system may include a first apparatus including a first substrate defining a first cavity and a first scaffold disposed within the first cavity. The first substrate and the first scaffold may collectively define a first set of channels including a first channel and a second channel. The first channel may be configured to receive and culture a first cell sample during use. The second channel may be configured to receive a fluid during use. The first scaffold may be configured to permit diffusion of the fluid through the first scaffold and into the first channel. A second apparatus may include a second substrate defining a second cavity and a second scaffold disposed within the second cavity. The second substrate and the second scaffold may collectively define a second set of channels including a third channel and a fourth channel, the third channel configured to receive and culture a second cell sample during use, the fourth channel configured to receive the fluid during use from the first apparatus, the second scaffold configured to permit diffusion of the fluid through the second scaffold and into the third channel. A set of fluid pumps may be coupled to one or more of the first set of channels and the second set of channels. A set of fluid sources may be coupled to the set of fluid pumps. Each fluid source may be coupled between a corresponding fluid pump and a corresponding channel of the first set of channels or the second set of channels. The first channel and the third channel may be configured to prohibit directly receiving fluid flow from the fluid pumped by the set of fluid pumps.

In some embodiments, a radiation source may be configured to emit a light signal that illuminates one or more of the first cell sample and the second cell sample. A detector may be configured to receive the light signal reflected from one or more of the first cell sample and the second cell sample. A controller may be coupled to the detector and include a processor and memory. The controller may be configured to receive signal data corresponding to the light signal received by the detector. Cell sample data may be generated using the signal data. In some embodiments, one or more phenotypes of the one or more first cell sample and the second cell sample may be identified using the cell sample data. The phenotypes may include at least one of non-invasive cells, invasive cells, size, shape, location, volume, growth rate, cell morphology, migration speed, DNA, RNA, and protein. In some embodiments, the radiation source may include one or more of a light emitting diode, laser, flash lamp, and optical fiber. The detector may comprise one or more of a lens, camera, measurement optics, optical sensor, charged coupled device, complementary metal-oxide semiconductor sensor, and filter.

In some embodiments, the set of fluid pumps includes a first pump coupled to the second channel and the fourth channel. The first set of channels may include a fifth channel. The set of fluid pumps may include a second pump coupled to the fifth channel. The first channel and the second channel may have a center-to-center distance of between about 1 mm and about 5 mm. A set of first anchors may be coupled to an interface between the first substrate and the first scaffold. The set of first anchors may include a first anchor attached to the first channel and a second anchor attached to the second channel. A length of a portion of the first anchor disposed wholly in the first channel is greater than a length of a portion of the second anchor disposed wholly in the second channel. The first cavity and the second cavity may be enclosed and sealed from an external environment. In some embodiments, ends of the first channel and the third channel are closed. In some embodiments, the first apparatus defines a set of first vents in the first scaffold, the set of first vents coupled to the first channel to allow air to escape when the first cell sample is applied to the first channel. In some embodiments, each channel of the first set of channels are on a same plane. In some embodiments, at least one channel of the first set of channels is on a different plane than the other channels of the first set of channels. In some embodiments, the second pump is configured to pump fluid through the fifth channel for a set of predetermined time periods. In some embodiments, the fluid flowing in the second channel is a first fluid, and the fluid flowing in the fifth channel is a second fluid different from the first fluid. In some embodiments, the fifth channel is configured to prohibit external fluid flow through the fifth channel after applying the first cell sample to the first channel. In some embodiments, at least one of the first fluid and the second fluid is a growth medium. In some embodiments, the fluid flowing through the second channel and the fifth channel is in a same direction. In some embodiments, the fluid flowing through the second channel and the fifth channel is in an opposite direction.

In some embodiments, one or more of the first cell sample and the second cell sample has a volume of at least about 20 μL. In some embodiments, the first channel is configured to receive one or more of phosphate buffered saline and fetal bovine serum. In some embodiments, one or more of the first cell sample and the second cell sample includes one or more of cancer cells including prostate cancer cells. In some embodiments, each channel of the first set of channels has a diameter of between about 0.2 mm and about 2.0 mm. In some embodiments, one or more of the first channel and the third channel is configured to receive between about 10⁷ cells and about 10⁵ cells. In some embodiments, the first substrate may be composed of one or more of polysiloxane, polydimethylsiloxane, polystyrene, polyethylene, and polycarbonate. In some embodiments, the first scaffold may be an extracellular matrix. In some embodiments, the extracellular matrix may be composed of one or more of collagen, hydrogel, agarose, polyethylene glycol, alginate, hyaluronan acid, laminin, fibronectin, proteoglycans, and elastin. In some embodiments, the collagen may include a concentration between about 1 mg/ml and about 10 mg/ml.

In some embodiments, the first substrate may be composed of polydimethylsiloxane in a 11:1 polymer-to-cross-linker volume ratio. The first substrate and the second substrate may be composed of polydimethylsiloxane in a polymer-to-cross-linker volume ratio between about 10:1 to about 30:1. In some embodiments, the set of fluid sources may be configured to hold an air bubble generated by the set of fluid pumps. The fluid may flow through the second channel and the fourth channel in a closed loop path. A third apparatus may be fluidically coupled in series with the first apparatus. The third apparatus may be fluidically coupled in parallel with the second apparatus.

In some embodiments, an apparatus may include a substrate defining a cavity and a scaffold disposed within the cavity. The substrate and the scaffold may collectively define a set of channels including a first channel and a second channel. The first channel may be configured to receive and culture a cell sample during use. The second channel may be configured to receive a fluid during use. The scaffold may be configured to permit diffusion of the fluid through the scaffold and into the first channel. A set of anchors may span an interface between the substrate and the scaffold, each anchor of the set of anchors disposed in a corresponding channel of the set of channels. In some embodiments, a length of a portion of the first anchor disposed wholly in the first channel is greater than a length of a portion of the second anchor disposed wholly in the second channel.

In some embodiments, the first channel may be configured without external fluid flow during the culture. The set of channels may be parallel. The first channel and the second channel may have a center-to-center distance of between about 1 mm and about 5 mm. In some embodiments, the second channel may include an anchor spanning an interface between the substrate and the scaffold. In some embodiments, the set of channels includes a third channel having a center-to-center distance from the first channel of between about 1 mm and about 5 mm. In some embodiments, each channel of the set of channels are on a same plane. In some embodiments, at least one channel of the set of channels is on a different plane than the other channels of the set of channels. In some embodiments, the fluid received in the second channel is a first fluid, and the third channel is configured to receive a second fluid different from the first fluid. In some embodiments, the third channel may be configured without flow of a fluid.

In some embodiments, at least one of the first fluid and the second fluid may be a growth medium. In some embodiments, the second channel and the third channel may be independently configured to receive the fluid in a predetermined direction. In some embodiments, one or more of the second channel and the third channel includes a valve. In some embodiments, the first channel may be configured to receive the cell sample having a volume of at least about 20 μL. In some embodiments, the first channel may be configured to receive between about 10⁷ cells and about 10⁵ cells. The cell sample may include one or more of cancer cells including prostate cancer cells. In some embodiments, each channel of the set of channels may have a diameter of between about 0.2 mm and about 2.0 mm. In some embodiments, the substrate may include a height of between about 3 mm to about 20 mm, a length of between about 10 mm and about 30 mm and a width of between about 1 mm and about 20 mm. In some embodiments, the substrate may be composed of one or more of polysiloxane, polydimethylsiloxane, polystyrene, polyethylene, and polycarbonate. The scaffold may be an extracellular matrix.

In some embodiments, the extracellular matrix may be composed of one or more of collagen, hydrogel, agarose, polyethylene glycol, alginate, hyaluronan acid, laminin, and fibronectin. The collagen may have a concentration of between about 1 mg/ml and about 2.5 mg/ml. In some embodiments, the substrate may be composed of polydimethylsiloxane in a 11:1 polymer-to-cross-linker volume ratio. The second channel may be configured to receive the fluid at a rate of between about 1 μL/min and about 200 μL/min for a predetermined time period. In some embodiments, the predetermined time period may be between about 1 day and about 28 days. In some embodiments, the fluid may be received continuously. A portion of the scaffold between the first channel and the second channel may have a pressure of between about 0 Pa and about 200 Pa during use. A portion of the scaffold between the first channel and the third channel may have a pressure of between about 0 Pa and about 200 Pa during use. The substrate may define a set of openings. A pressure sensor may be coupled to the substrate.

### BRIEF DESCRIPTION OF THE DRAWINGS

FIG. 1A is an illustrative cross-sectional plan view of a cell culture system in a first configuration, according to embodiments. FIG. 1B is another cross-sectional plan view of the cell culture system depicted in FIG. 1A in a second configuration. FIG. 1C is yet another cross-sectional plan view of the cell culture system depicted in FIG. 1A in a third configuration.

FIG. 2A is an illustrative cross-sectional plan view of a cell culture system, according to embodiments. FIG. 2B is an illustrative block diagram of another cell culture system, according to embodiments.

FIG. 3A is an illustrative plan view of a cell culture device, according to other embodiments. FIG. 3B is another illustrative plan view of a portion of the cell culture device depicted in FIG. 3A. FIG. 3C is an illustrative perspective view of the cell culture device depicted in FIG. 3B. FIG. 3D is a side view of the cell culture device depicted in FIG. 3B. FIG. 3E is another illustrative plan view of another portion of the cell culture device depicted in FIG. 3A. FIG. 3F is an illustrative perspective view of an anchor of the cell culture device depicted in FIG. 3A.

FIG. 4 is a plan view of a step in a manufacturing process of an extracellular matrix of a cell culture device, according to embodiments.

FIG. 5 is a plan view of a step in a manufacturing process of a set of channels of a cell culture device, according to embodiments.

FIG. 6 is a perspective view of a step in a manufacturing process of a cell culture device, according to embodiments.

FIG. 7A is a cross-sectional plan view of a fluid source, according to embodiments. FIG. 7B is an exploded side view of the fluid source depicted in FIG. 7A.

FIGS. 8A-8B are block diagrams of a cell culture system, according to other embodiments.

FIG. 9 is an illustrative flowchart of a method of using a microfluidic cell culture device, according to embodiments.

FIG. 10 is an illustrative flowchart of another method of using a microfluidic cell culture device, according to embodiments.

FIG. 11 is an illustrative flowchart of a method of manufacturing a microfluidic cell culture device, according to embodiments.

FIG. 12A is an illustrative fluid flow vector and interstitial fluidic pressure diagram of a cell culture device, according to embodiments. FIG. 12B is an illustrative graph depicting the interstitial pressure in the cell culture device depicted in FIG. 12A.

FIGS. 13A-13C are illustrative graphs depicting cell growth over time. FIG. 13A illustrates a migration distance of PC3 cells in different regions of the cell culture device. FIG. 13B illustrates a migration distance of DU145 cells in different regions of the cell culture device FIG. 13C illustrates an overall migration distance of PC3 cells relative to DU145 cells.

FIGS. 14A-14B are illustrative graphs depicting cell growth as a function of fluid flow rate and extracellular matrix concentration. FIG. 14A illustrates a migration distance of PC3 cells as a function of location, flow rate, and concentration. FIG. 14B illustrates a migration distance of DU145 cells as a function of location, flow rate, and concentration.

FIG. 15A depicts images of stained PC3 cells. FIG. 15B depicts images of stained DU145.

FIG. 16A is an image of an extracted tumoroid having invasive and non-invasive cells after cell culture in a cell culture device, according to embodiments. FIG. 16B is an image of the extracted tumoroid depicted in FIG. 16A after enzymatic dissociation. FIG. 16C is a graph of real-time polymerase chain reaction (q-RT-PCR) analysis of target gene Ki-67 expression relative to RPL22 reference gene of the sample imaged in FIG. 16A.

### DETAILED DESCRIPTION

Described herein are embodiments of cell culture devices, systems, and methods of use thereof. These systems and methods may be used to characterize and/or quantitate a three-dimensionally cultured cell sample and permit spatiotemporal analysis of leader cells at a tumor invasion front. For example, the devices and systems described herein may be used to three-dimensionally culture a cell sample in a sealed environment subject to interstitial flow and/or interstitial pressure, which may be experienced by invading cancer cells in vivo by blood and/or lymphatic flow. For example, tumor cells in a human body may be subject to mechanical forces such as interstitial pressure, compressive stress, shear stress, and stiffness. However, the effect of external sources of interstitial pressure applied indirectly to tumor cells are not well understood and may be modeled and analyzed using the devices, systems, and methods described herein where, for example interstitial pressure may be applied continuously from an adjacent fluidic channel.

The cell sample may be cultured over a predetermined period of time (e.g., about 21 days or more) to permit analysis of, for example, non-invasive cancer cells and invasive cancer cells. For example, invading cells and non-invading cells from the same biological sample may be isolated and analyzed separately for statistical and biological analysis and comparison. In some embodiments, invading and non-invading cells may be demarcated by visual observation under a microscope where invading cells invade or migrate into the extracellular matrix and non-invading cells remain within a channel. By contrast, a whole tumor may be conventionally analyzed for biomarkers without regard to a cell subpopulation (e.g., invading and non-invading). The devices, systems, and methods described herein may permit one or more of analysis of tumor invasion at a single cell level, analysis of a three-dimensional invasion front of a tumor mass or organoid grown in a three-dimensional matrix, as well as for high-throughput drug screening. For example, the cell sample may be imaged and/or extracted in order to identify one or more phenotypes of the cultured cell sample.

Each of the devices (101, 201, 211, 251, 252, 253, 300, 302, 304, 306, 308, 400, 500, 600, etc.) described in detail herein may receive a cell sample including, but not limited to, mammalian cells including cancer cells, epithelial cells, fibroblasts, immune cells, endothelial cells, and nerve cells. The cancer cells may include prostate cancer cells. Any of the devices (101, 201, 211, 251, 252, 253, 300, 302, 304, 306, 308, 400, 500, 600, etc.) as described herein may be used with a suitable cell culture system and method. Any number of analytical methods may be adapted for use in the cell culture devices disclosed herein, depending upon the particular phenotype and sample being analyzed.

Some cell types are typically found spatially arranged in a sealed, three-dimensional environment (e.g., within a human or animal body) and are subject to a variety of mechanical and fluid dynamic forces such as interstitial flow, interstitial pressure or shear stress, under laminar and turbulent flow. The devices, systems, and methods described herein may be configured to provide one or more of these forces during long-term cell culture (e.g., weeks, months) in a sealed three-dimensional environment, and may be used to model normal or malignant cell behavior such as carcinogenesis, invasion, or wound healing. The cell culture may be analyzed in a spatiotemporal manner. For example, single cell imaging of dynamic processes such as invasion may be used to analyze cell subsets and/or molecules thereof (e.g., molecules of cells originating from the invasion front of a tumor).

### I. Devices

Described herein are devices that may be used in some embodiments of the various systems described. A microfluidic cell culture device as described herein may include a substrate defining a cavity and a scaffold disposed within the cavity. The scaffold may be, for example, an extracellular matrix that may define a set of parallel channels. In some embodiments, a cell sample may be applied and cultured within a first channel of the set of channels. For example, a cell sample such as tumor cells DU145 and/or PC3 may be seeded in the first channel to form a tumoroid of about 500 μm in diameter. The cell sample may be mammalian and may be grown three-dimensionally in a sealed environment to aid cell growth and invasion into the scaffold. As used herein, mammalian cells may include one or more cancer cells, epithelial cells, fibroblasts, immune cells, endothelial cells, and nerve cells. This may allow a cancerous cell sample to metastasize and be analyzed at predetermined time intervals.

In some embodiments, the cell culture device may define a second channel coupled to a fluid pump and/or fluid source to form a closed-loop fluid circuit for moving fluid through the second channel of the cell culture device. Fluid flow through the set of channels may be modulated to control physical, chemical, and biological factors (e.g., interstitial flow, media flow rate) that effect cell growth. During use, direct external fluid flow is prohibited through the first channel. In some embodiments, continuous fluid flow may be provided at least through the second channel for a predetermined period of time (e.g., about 21 days) such that the first channel is subject to indirect interstitial pressure from the fluid flowing through the second channel. While fluid may diffuse from the second channel into the first channel, a direct fluid flow path is not established between the ends of the first channel. This would, for example, allow the study of the effects of interstitial pressure on the behavior of small avascular tumors, which are typically exposed to external interstitial pressure arising from the blood and lymphatic circulatory system, and may provide insight into early tumor biology such as early disseminated tumor cells. The effect of interstitial pressure may be controlled without having to consider additional confounding factors that may arise if fluid flow and pressure originated from the first channel bearing cells. For example, the ends of the first channel may be closed off using a set of plugs.

In some embodiments, the growth of the cell culture may be imaged and analyzed at predetermined time intervals (e.g., 1, 5, 12, 15, 18, 22, 28, 60 days, and all intervals in between). For example, invasion distance of the cell sample may be measured relative to the second channel and the third channel. As such, recapitulation of the tumor invasion front may allow quantification of invasive potential over time. In some embodiments, one or more pharmacological agents may be applied to a cell sample and analyzed over time.

A third channel may be disposed adjacent to the first channel and opposite the second channel. In some embodiments, the third channel may be used as a control that does not receive fluid flow. In some other embodiments, the third channel may have a similar configuration to the second channel and be used to move fluid through the third channel either in the same or opposite direction as the second channel. In still other embodiments, the set of channels may include additional channels such as a fourth, fifth, sixth, and seventh channel, and so forth. In some of these embodiments, a plurality of the channels of the set of channels may be configured to receive a cell sample, which may be the same or different cell sample.

The apparatus may be configured to be used with a cell culture system to quantify and analyze characteristics of the cell sample over time. For example, optical measurements of the cell sample may be performed at predetermined time intervals to identify one or more phenotypes of the cell sample. As another example, the cell sample may be enzymatically dissociated to separate the non-invasive cancer cells and invasive cancer cells for analysis.

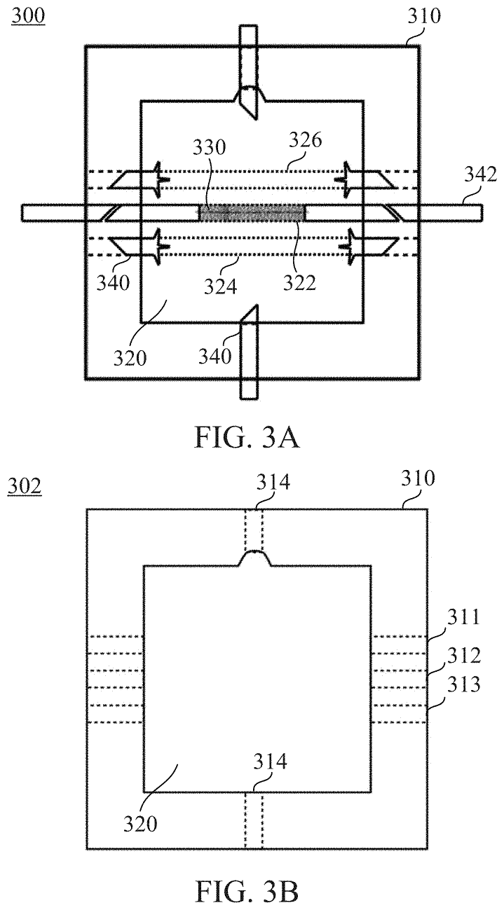

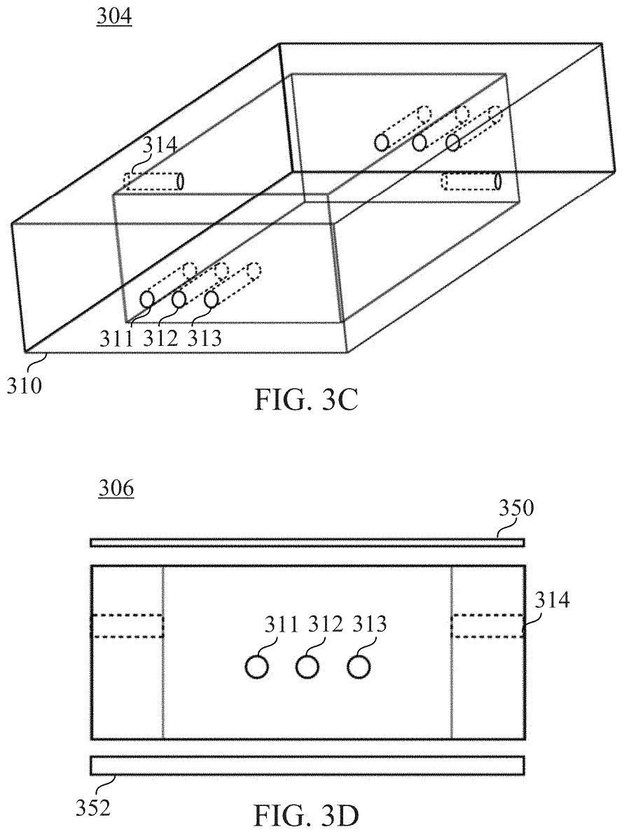

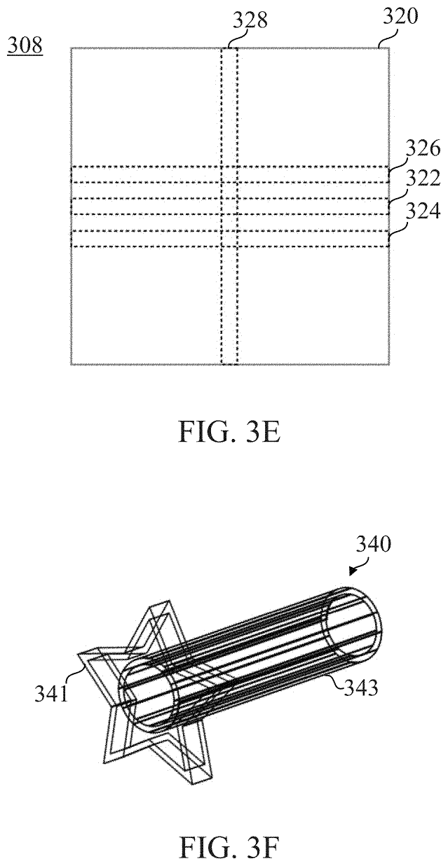

FIG. 3A is an illustrative plan view of a cell culture device (300). The cell culture device (300) may be used for continuous culture of a cell sample (e.g., mammalian cells) in a sealed three-dimensional system for a predetermined period of time (e.g., about 21 days). As described in more detail herein, a cell sample (330) may be immobilized in a polymerized scaffold (320). A continuous flow of growth medium (e.g., first fluid, second fluid) may be maintained through the scaffold (320). In some embodiments, a flow rate and a pressure of the fluid may be varied. Changes in one or more of an interstitial flow rate, interstitial pressure on the cells, cell growth, cell death, and cell migration may be identified and analyzed, as described in more detail herein.

The device (300) may include a substrate (310) defining a cavity in which a scaffold (320) is disposed. The substrate (310) and the scaffold (320) may collectively define a set of channels (322, 324, 326)) including a first channel (322) and a second channel (324). The first channel (322) may be configured to receive and culture a cell sample (330) during use. The second channel (324) may be configured to receive a fluid (not shown) during use. The scaffold (320) may be configured to permit diffusion of the fluid through the scaffold (320) and into the first channel (322) from any of the other channels of the set of channels. The first channel (322) may be configured without external fluid flow during the culture of the cell sample (330). For example, a set of plugs (342) may be coupled to the ends of the first channel (322) to prohibit external fluid flow from an external source such as a fluid pump and reservoir. That is, external fluid flow is prohibited through the first channel after applying the cell sample to the first channel such that the first channel is subject to indirect interstitial pressure from the fluid flowing through adjacent channels. The set of plugs (342) may be removably attached so as to allow input of a discrete volume to be introduced into the first channel (322). For example, a plug (342) may be temporarily removed from an end of the first channel (322) to introduce one or more of phosphate buffered saline, fetal bovine serum, and cell sample.

As shown in FIG. 3A, the set of channels (322, 324, 326) may be parallel. In some embodiments, the first channel (322) and the second channel (324) may have a center-to-center distance of between about 1 mm and about 5 mm. The set of channels (322, 324, 326) may include a third channel (326) having a center-to-center distance from the first channel (322) of between about 1 mm and about 5 mm. In some embodiments, the third channel (326) may be configured to receive a fluid. In some of these embodiments, the fluid received in the second channel (324) is a first fluid, and the fluid received in the third channel (326) is a second fluid different from the first fluid. At least one of the first fluid and the second fluid may be a growth medium. The second channel (324) and the third channel (326) may be independently configured to receive the fluid in a predetermined direction. In other embodiments, the third channel (326) may be configured without flow of a fluid. For example, one or more of the second channel (324) and the third channel (326) may include a valve (not shown) that may control fluid flow including flow rate and direction.

In some embodiments, each channel of the set of channels (322, 324, 326) may be on a same plane. In other embodiments, at least one channel of the set of channels (322, 324, 326) may be on a different plane than the other channels of the set of channels. In some embodiments, each channel of the set of channels (322, 324, 326) may have a diameter of between about 0.2 mm and about 2.0 mm.

As described in more detail herein, the set of channels (322, 324, 326) may include a set of anchors (340) that attach to an interface between the substrate (310) and the scaffold (320). For example, the second channel (324) and third channel (326) may each include a pair of anchors (340) spanning an interface between the substrate (310) and the scaffold (320). The set of anchors (340) may be configured to provide support and physical reinforcement to the scaffold disposed in the substrate. For example, the anchors (340) spanning the interface between the substrate (310) and the scaffold (320) corresponding to the second channel (324) and the third channel (326) may help maintain the structural integrity of those channels (324, 326) in those areas subject to relatively high stress forces due to fluid flow. As described in more detail herein, the shape of the anchors (340) may be optimized for secure attachment to the substrate (320). Furthermore, the set of anchors (340) may provide a barrier within its channel to prevent fluid flow along an interface between the substrate (310) and the scaffold (320) (e.g., a space between an inner sidewall of the substrate (310) and outer wall of the scaffold (320)). That is, the set of anchors (340) may ensure that fluid flow enters the substrate (310) through a respective channel.

In some embodiments, a length of an anchor of a first channel (322) may be longer than a length of an anchor of the second channel (324) and third channel (326). For example, the anchors may have a difference in length of between about 5 mm and about 20 mm. Within the scaffold, interstitial pressure is highest at the interface between the scaffold and substrate or at an end of an anchor. In order to prevent the cell sample in the first channel from experiencing excessive interstitial pressure and to more evenly distribute the interstitial pressure, the anchors of the cell sample channel and the fluid channels may differ in length as described herein.

In some embodiments, the first channel (322) may be configured to receive the cell sample (330) having a volume of at least about 20 μL. The first channel (322) may be configured to receive between about 10⁷ cells and about 10⁵ cells. The cell sample (330) may include one or more of mammalian cells and cancer cells including prostate cancer cells. In some embodiments, fluid may flow through the device (300) between about 1 day and about 60 days. In some embodiments, fluid may flow through the device (300) between about 21 days and about 28 days. In some of these embodiments, the fluid may be received continuously or substantially continuously. In some embodiments, a portion of a scaffold (320) between the first channel (322) and the second channel (324) may have a pressure of between about 0 Pa and about 200 Pa during use. In some embodiments, a portion of the scaffold between the first channel (322) and the third channel (326) may have a pressure of between about 0 Pa and about 200 Pa during use.

In some embodiments, the substrate (310) may include a height of between about 3 mm to about 20 mm, a length of between about 10 mm and about 30 mm, and a width of between about 1 mm and about 20 mm. The substrate (310) may be composed of one or more of polysiloxane, polydimethylsiloxane, polystyrene, polyethylene, and polycarbonate. In some embodiments, the substrate (310) may be composed of polydimethylsiloxane polymer-to-cross-linker volume ratio between about 10:1 to about 30:1. In some embodiments, the substrate (310) may be composed of polydimethylsiloxane in a 11:1 polymer-to-cross-linker volume ratio.

As shown in the cell culture devices (302, 304, 306) in respective FIGS. 3B-3D, the substrate (310) may define a set of openings (311, 312, 313, 314) through which the set of anchors (340) and plugs (342) (not shown in FIGS. 3B-3D) may be disposed. In FIG. 3C, the set of openings (311, 312, 313, 314) surround and open into a hollow interior (e.g., cavity) of the substrate (310). A subset (312) of the set of openings may be configured to allow liquid to escape the first channel (322) when a cell sample (330) is applied in the first channel (322).

FIG. 3E is a plan view of a scaffold (308) of the cell culture device (300) depicted in FIG. 3A. The scaffold (308) includes a first channel (322), a second channel (324), and a third channel (326). The scaffold (308) may include one or more vent channels (328) that may be substantially perpendicular to the set of channels (322, 324, 326). As shown in FIG. 3E, a vent channel (328) may be disposed perpendicular to the set of channels (322, 324, 326) and be configured to allow air to vent out of the scaffold (320) and subsequently through an opening (314) in the substrate (310).

Although the substrate (310) and hollow interior are both shown as rectangular, they may form any desired shape such as spherical, elliptical, oblong, polygonal, and the like. As shown in the cell culture device (306) depicted in FIG. 3D, opposing sides of the substrate (310) may be coupled to a first transparent layer (350) and a second transparent layer (352) such that the cell culture device (306) may form a sealed chamber and closed-loop system once the anchors (340), plugs (342), connectors, fluid pump, and fluid reservoir are coupled to the device. That is, the system may be sealed from an external environment such that an interstitial pressure may be formed in the scaffold (320) due to the flow of fluid through the system rather than due to the external environment. A sealed cavity ensures a consistent pressurized system, which an unsealed cavity may not be able to properly achieve. In addition, a sealed cavity may reduce the amount of fluid loss due to water evaporation, which would otherwise affect ionic concentration within the scaffold. In some embodiments, the substrate may be gas permeable and hydrophobic, thereby allowing gas exchange but preventing water loss by repelling water from crossing the substrate.

In some embodiments, the scaffold (308) may be an extracellular matrix. The extracellular matrix may be composed of one or more of collagen, hydrogel, agarose, polyethylene glycol, alginate, hyaluronan acid, laminin, fibronectin, proteoglycans, and elastin. In some embodiments, the collagen may have a concentration of between about 1 mg/ml and about 10 mg/ml. In some embodiments, the collagen may have a concentration of between about 1 mg/ml and about 2.5 mg/ml. This wide and dynamic range of collagen and/or extracellular matrix protein concentration is a factor in extracellular matrix stiffness and composition that corresponds to the stiffness of different tissues and organs in the body range from very soft (e.g., brain) to hard (e.g., bone).

FIG. 3F is a perspective view of an anchor (340) of the cell culture device (300) depicted in FIG. 3A. The anchor (340) may generally have a size and shape (e.g., tubular) that allows it to be inserted into a channel of the set of channels. A first end (341) of the anchor (340) may include a set of protrusions configured to engage and secure to the scaffold (320). For example, the first end (341) may be flared and include a set of barbs, spikes, hooks, combinations thereof, and the like. FIG. 3F illustrates the first end (341) with a star-like pattern. Although not depicted in FIG. 3F, a second end (343) of the anchor (340) opposite the first end (341) may include an angled and/or beveled shape configured to ease insertion into an opening in the substrate (310). In some embodiments, the anchor (340) may have a diameter of between about 0.5 mm and about 2.0 mm and a length of between about 3.0 mm and about 15 mm. The set of anchors (340) and set of plugs (342) may be composed of any of the materials described with respect to the substrate (310). In some embodiments, the set of plugs (342) may be composed of PDMS in a 11:1 polymer to cross-linker volume ratio.

### II. Systems

### Cell Culture System

Described herein are cell culture systems that may include one or more of the components necessary to culture a cell sample using the devices described herein. For example, the cell culture systems described herein may automatically support, grow, image, and analyze a cell sample applied to a cell culture device. Generally, the cell culture systems described herein may include one or more of a cell culture device, a fluid pump, a fluid source, and a controller (including memory, a processor, and computer instructions). The cell culture device may include a sealed chamber defining a cavity filled with a scaffold for immobilizing a cell sample (e.g., mammalian cells). A set of fluidic channels may be defined within the sealed chamber. At least one of the channels may be configured to receive immobilized mammalian cells. A pump may be coupled to at least one channel of the set of channels. The pump may be configured to provide fluid flow of a culture medium (e.g., growth medium) within the scaffold. A controller coupled to the pump may be configured to vary the fluid flow rate and pressure of the culture medium. In some embodiments, the system may be configured to continuously culture the mammalian cells embedded in a sealed three-dimensional environment for at least 21 days. The cell culture may be imaged and/or extracted from the system for analysis.

In some embodiments, a set of cell culture devices may be configured in series and/or parallel where the same and/or different cell samples may be applied to a channel of each cell culture device. Cell culture devices in series may allow a cell sample cultured over time in an upstream device to be fluidically transported into a downstream cell culture device where the phenotypes and interactions between the connected cell culture devices may be analyzed. As not all invasive cells may proceed to form metastatic lesions, some of these embodiments further allow the benefit of separating invasive cells that may form metastatic lesions from the general invasive cell population. These invasive cells that may form metastatic lesions may invade into the scaffold (e.g., collagen, extracellular matrix) of the downstream cell culture device whereas invasive cells that invaded in the upstream cell culture device that do not form metastatic lesions will be collected in the reservoir.

In some embodiments, the system may include a radiation source configured to emit a light signal (e.g., light beam) that illuminates the cell sample. A detector may be configured to receive the light signal reflected from the cell sample. A controller coupled to the detector may be configured to receive signal data corresponding to the light signal received by the detector and generate cell sample data using the signal data. One or more analytes of the fluid may be identified by the controller using the analyte data. One or more phenotypes of the cell sample may be identified using the cell sample data.

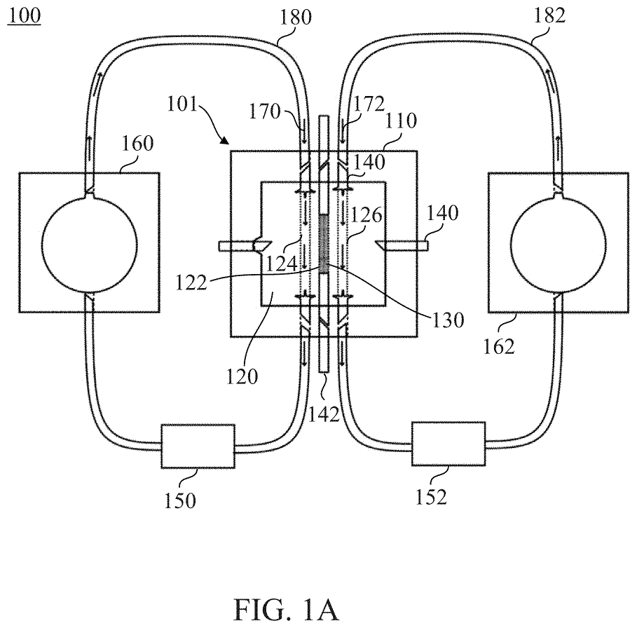

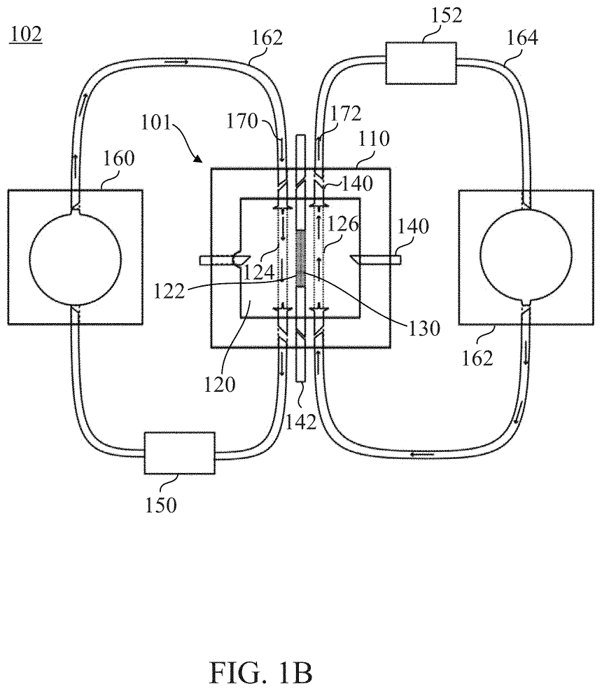

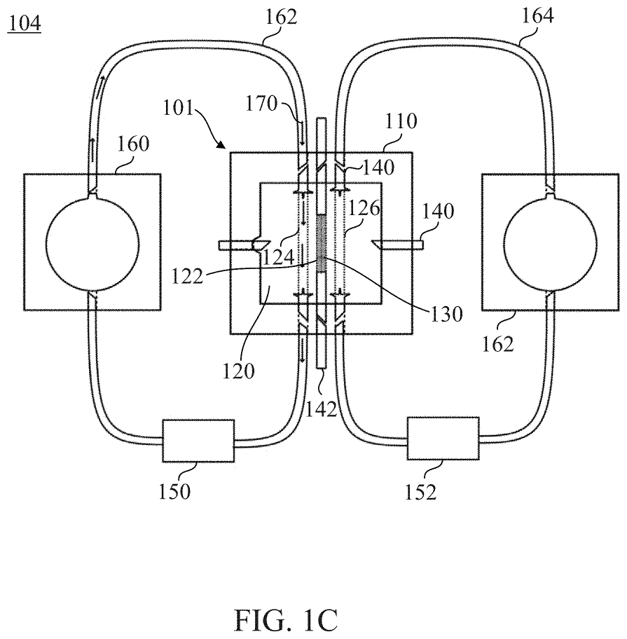

FIG. 1A is an illustrative cross-sectional plan view of a cell culture system (100) in a first configuration. The system (100) may include a cell culture device (100) coupled to a set of pumps (150, 152) and fluid sources (160, 162) (e.g., reservoirs). The device (101) may include a substrate (110) defining a cavity in which a scaffold (120) is disposed. The substrate (110) and the scaffold (120) collectively define a set of channels (122, 124, 126) including a first channel (122) and a second channel (124). The first channel (122) may be configured to receive and culture a cell sample (130) during use. In some embodiments, the first channel (120) may be configured to receive one or more of phosphate buffered saline and fetal bovine serum. The second channel (124) may be configured to receive a fluid (170) during use. The scaffold (120) may be configured to permit diffusion of the fluid through the scaffold (120) and into the first channel (122) from any of the other channels of the set of channels. The first channel (122) may be configured without external fluid flow during the culture of the cell sample (130). For example, a set of plugs (142) may be coupled to the ends of the first channel (122) to prohibit external fluid flow from an external source such as a fluid pump (150, 152) and fluid sources (160, 162). Fluid (170, 172) may flow into the first channel (122) through the scaffold (120) over time.

A set of fluid pumps (150, 152) may be coupled to one or more channels of the set of channels (122, 124, 126). The set of fluid pumps (150, 152) may include one or more peristaltic pumps. For example, a first fluid pump (150) may be coupled to the second channel (124) and a second fluid pump (152) may be coupled to the third channel (126). A set of fluid sources (160, 162) may be coupled to the set of fluid pumps (150, 152). For example, the first fluid pump (150) may be coupled to a first fluid source (160) and the second fluid pump (152) may be coupled to a second fluid source (162). Furthermore, a first connector (180) (e.g., tube) may couple the first fluid pump (150) and the first fluid source (160) to each end of the second channel (124).

Likewise, a second connector (182) may couple the second fluid pump (152) and the second fluid source (162) to each end of the third channel (164). In the first configuration, fluid flows (170, 172) in the same direction through the second channel (124) and third channel (126). That is, the outlet of the second channel (124) and the third channel (126) may be coupled to an input of respective fluid pumps (150, 152). In some embodiments, the set of fluid sources (160, 162) may be formed of the same substrate material as the cell culture device (101). The set of fluid sources (160, 162) may be configured to store fluid (e.g., cell growth media, therapeutic agent, pharmacological agent, drug, and combinations thereof). The set of fluid pumps (150, 152) may be configured to recirculate the fluid (170, 172) through a closed circuit (closed loop path) of the system (100).

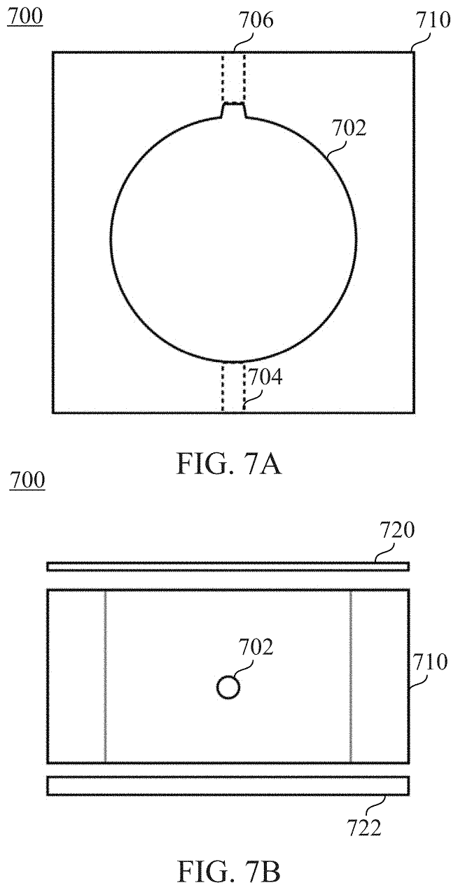

As shown in FIG. 1A, for example, a fluid source (160) may be coupled between a fluid pump (150) and an inlet of the device (101). In some embodiments, the fluid source (160) may be configured to hold an air bubble generated by the fluid pump (150) and thus prohibit formation of an air bubble through the second channel (124). That is, air bubbles may be held in a fluid source upstream of a channel. For example, the fluid source (160) may contain a cavity configured to hold a volume of fluid where the fluid fills the cavity up to a predetermined height that is above an inlet height and outlet height of the fluid source (160) (see FIG. 7B). The space above the predetermined height in the cavity of the fluid source (160) may contain air. During use, operation of the pump (150) may generate one or more air bubbles that may be passed through the connector (180) and into the fluid source (160). When the air bubble passes into the fluid source (160), the air within the bubble may be held within the fluid source (160) such that fluid without air bubbles may be output from an outlet of the fluid source (160) and into the device (101). Thus, air bubbles may be prohibited from forming in the channels of the device (101). An additional fluid source may also be disposed downstream of the second channel (124) and configured to capture any invasive cells that entered the circulation through the second channel (124) and into fluid flow.

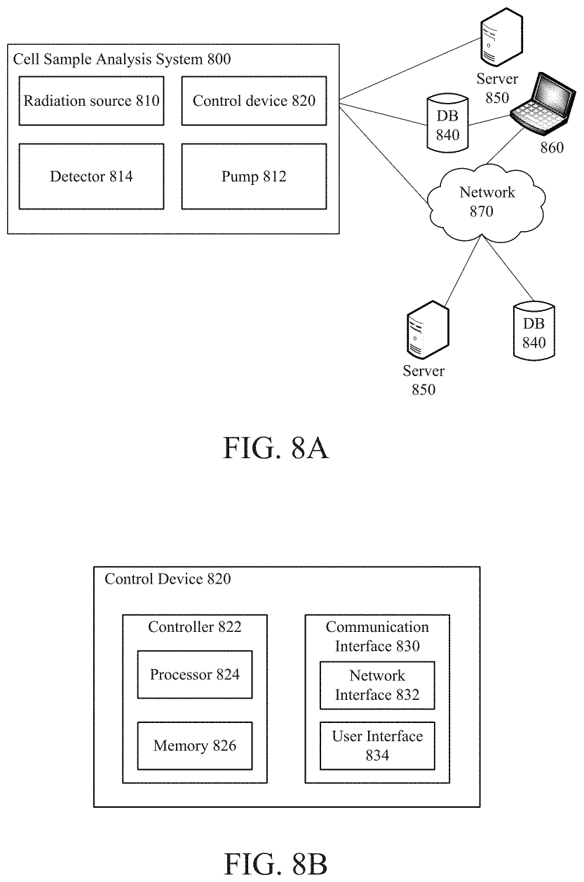

As shown in the block diagram of FIG. 8A, a radiation source (810) may be configured to emit a light signal that illuminates the cell sample (130). The radiation source (810) may include one or more of a light emitting diode, laser, flash lamp, optical fiber, combinations thereof, and the like. A detector (814) may be configured to receive the light signal reflected from the cell sample (130). The detector (814) may include one or more of a lens, camera, measurement optics, optical sensor, charged coupled device, complementary metal-oxide semiconductor sensor, filter, combinations thereof, and the like.

A controller (822) of a control device (820) may be coupled to the detector (814) and include a processor and memory. In some embodiments, the controller (822) may be configured to receive signal data corresponding to the light signal received by the detector (814), generate cell sample data using the signal data, and identify one or more phenotypes of the cell sample (130) using the cell sample data. In some embodiments, the phenotype may include at least one of non-invasive cells, invasive cells, size, shape, location, volume, growth rate, cell morphology, migration speed, DNA, RNA, and protein.

The set of channels (122, 124, 126) may be parallel. In some embodiments, the first channel (122) and the second channel (124) may have a center-to-center distance of between about 1 mm and about 5 mm. The set of channels (122, 124, 126) may include a third channel (126) having a center-to-center distance from the first channel (122) of between about 1 mm and about 5 mm. In some embodiments, the third channel (126) may be configured to receive a fluid. In some of these embodiments, the fluid received in the second channel (124) may be a first fluid, and the fluid received in the third channel (126) may be a second fluid different from the first fluid. At least one of the first fluid and the second fluid may be a growth medium. The second channel (124) and the third channel (126) may be independently configured to receive the fluid in a predetermined direction as described in more detail herein. In other embodiments, the third channel (326) may be configured without flow of a fluid (see FIG. 1C). For example, one or more of the second channel (124) and the third channel (126) may include a valve (not shown) that may control fluid flow including flow rate and direction.

In some embodiments, each channel of the set of channels (122, 124, 126) may be on a same plane. In other embodiments, at least one channel of the set of channels (122, 124, 126) may be on a different plane than the other channels of the set of channels. In some embodiments, each channel of the set of channels (122, 124, 126) may have a diameter of between about 0.2 mm and about 2.0 mm.

The set of channels (122, 124, 126) may include a set of anchors (140) that attach to an interface between the substrate (110) and the scaffold (120). For example, the second channel (124) and third channel (126) may each include a pair of anchors (140) spanning at least a portion of an interface between the substrate (110) and the scaffold (120). The substrate (110) may define a set of openings through which the set of anchors (140) and plugs (142) may be disposed. The set of anchors (140) may be configured to provide support and physical reinforcement to the scaffold disposed in the substrate. For example, the anchors (140) spanning the interface between the substrate (110) and the scaffold (120) corresponding to the second channel (124) and the third channel (126) may help maintain the structural integrity of those channels (124, 126) in those areas subject to relatively high stress forces due to fluid flow. As described in detail herein, a length of the anchors may differ.

In some embodiments, the first channel (122) may be configured to receive the cell sample (130) having a volume of at least about 20 μL. The first channel (122) may be configured to receive between about 10⁷ cells and about 10⁵ cells. The cell sample (130) may include one or more of mammalian cells and cancer cells including prostate cancer cells. In some embodiments, fluid may flow through the device (100) between about 1 day and about 28 days. In some of these embodiments, the fluid may be received continuously or substantially continuously. In some embodiments, a portion of a scaffold (320) between the first channel (122) and the second channel (124) may have a pressure of between about 0 Pa and about 200 Pa during use. In some embodiments, a portion of the scaffold between the first channel (122) and the third channel (126) may have a pressure of between about 0 Pa and about 200 Pa during use.

The substrate (110) may form a sealed chamber. In some embodiments, the substrate (110) may include a height of between about 3 mm to about 20 mm, a length of between about 10 mm and about 30 mm, and a width of between about 1 mm and about 20 mm. The substrate (110) may be composed of one or more of polysiloxane, polydimethylsiloxane, polystyrene, polyethylene, and polycarbonate. In some embodiments, the substrate (110) may be composed of polydimethylsiloxane in about a 11:1 polymer-to-cross-linker volume ratio. In some embodiments, one side of the chamber may include a transparent portion (e.g., glass) that allows imaging of the cell culture in the scaffold (120) to aid analysis of cell growth, invasion, and/or migration.

In some embodiments, the scaffold (120) may be an extracellular matrix. The extracellular matrix may be composed of one or more of collagen, hydrogel, agarose, polyethylene glycol, alginate, hyaluronan acid, laminin, fibronectin, proteoglycans, and elastin. In some embodiments, the collagen may have a concentration of between about 1 mg/ml and about 10 mg/ml.

FIG. 1B is another cross-sectional plan view of the cell culture system (102) in a second configuration. Elements of the system (102) sharing the same elements as those described with respect to the system (100) are not repeated. That is, any of the cell culture devices as described herein may be used in any of the systems described herein. In the second configuration, fluid flows (170, 172) in opposite directions through the second channel (124) and third channel (126). The outlets of the second channel (124) and the third channel (126) may be coupled to an input of respective fluid pumps (150, 152).

FIG. 1C is yet another cross-sectional plan view of the cell culture system (104) in a third configuration. Elements of the system (104) sharing the same elements as those described with respect to the system (100) are not repeated. In the third configuration, a fluid flows (170) in a first direction through the second channel (124) but fluid does not flow through the third channel (126). For example, the third channel (126) may be configured to prohibit external fluid flow (172) through the third channel (126) after applying the cell sample (130) to the first channel (122). The third channel (126) without externally sourced fluid flow may function as a control. The outlet of the second channel (124) may be coupled to an input of the first fluid pump (150).

In some embodiments, the set of fluid sources (160, 162) and set of connectors (180, 182) may be primed with the fluid (170, 172). Any gaseous bubbles that may form within the set of connectors (180, 182) may be flushed or aspirated using a fluid-filled syringe to apply positive or negative pressure. In some embodiments, the set of fluid sources (160 162) may receive the respective output of the set of pumps (150, 152). This system configuration may minimize the formation of gaseous bubbles. In some embodiments, the set of connectors (180, 182) may be tubes composed of PTFE that couple to each of the device (101), set of fluid pumps (150, 152), and set of fluid sources (160, 162) to form a closed-loop system (e.g., a sealed environment).

In some embodiments, a cell culture system may include a set of fluidically connected cell culture devices that may be used to model a metastatic process including one or more of invasion, intravasation, circulation of tumor cells, and downstream extravasation. For example, a first cell sample may be cultured within a first cell culture device as described herein. Over time, the cell sample may invade a fluidic channel and circulate through the system to one or more other cell culture devices located downstream of the first cell culture device. In some of these embodiments, the other cell culture devices may include a second cell sample (e.g., tissue) that may be configured to interact with the first cell sample. In this manner, the first cell sample may be configured to grow metastatic lesions in one or more downstream cell culture devices. Migrating cells of the first cell sample that flow through the other cell culture devices may be accumulated at a fluid source coupled to the cell culture devices. One application of such an embodiment may include isolating invasive cells based on ability to establish metastatic lesions at different tissue sites. For example, a first downstream cell culture device may be configured to grow bone cells while a second downstream cell culture device may be configured to grow lung cells. This may allow invasive cells to home to and form bone metastatic lesions may invade the first downstream cell culture device while invasive cancer cells may home to and form lung metastatic lesions in the second downstream cell culture device. The systems described herein may allow the isolation, separation, and analysis of invasive cancer cells based on their unique ability to further metastasize to specific tissue. As described herein, a controller may control a fluid flow rate through the closed-loop sealed system to modulate the various mechanical and fluid dynamic forces such as interstitial flow and interstitial pressure. Also, as described herein in more detail, cell subsets may be isolated and separated from each of the cell culture devices for analysis.

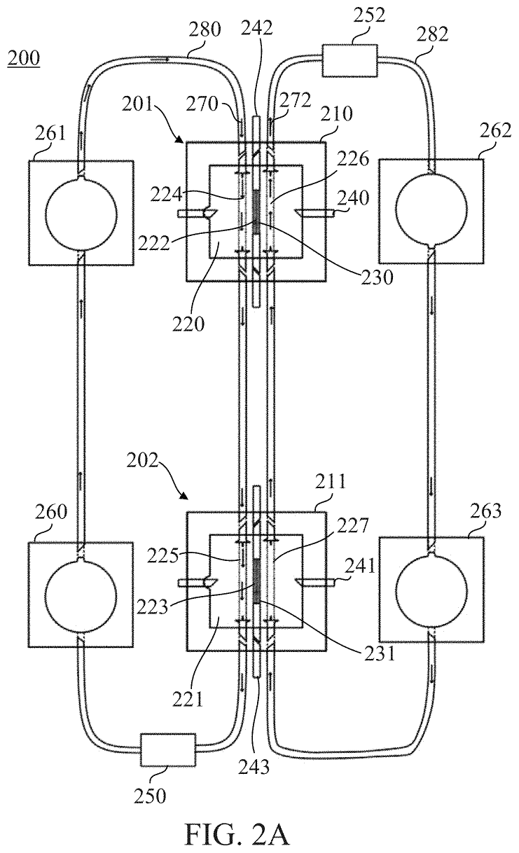

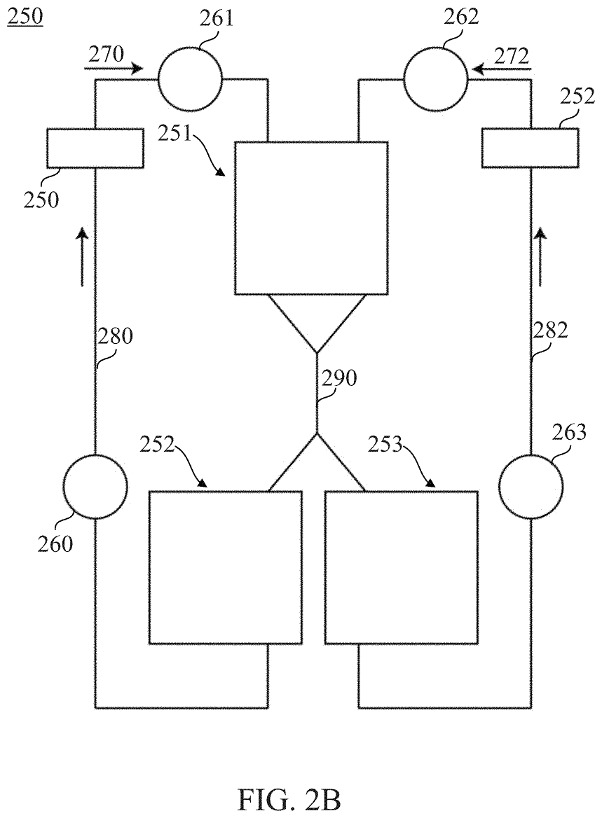

FIG. 2A is an illustrative cross-sectional plan view of a cell culture system (200) having a set of cell culture devices (201, 202) connected in series. The system (200) may include a first cell culture device (201) and a second cell culture device (202) each coupled to a set of pumps (250, 252) and a set of fluid sources (260, 261, 262, 263) (e.g., reservoirs). The first and second cell culture devices may include any of the elements of any of the cell culture devise as described herein. The first cell culture device (201) may include a first substrate (210) defining a cavity in which a first scaffold (220) is disposed. The first substrate (210) and the first scaffold (220) may collectively define a set of channels (222, 224, 226) including a first channel (222) and a second channel (224). The first channel (222) may be configured to receive and culture a cell sample (230) during use. In some embodiments, the first channel (222) may be configured to receive one or more of phosphate buffered saline and fetal bovine serum. The second channel (224) may be configured to receive a first fluid (270) during use. The first scaffold (220) may be configured to permit diffusion of the fluid through the first scaffold (220) and into the first channel (222) from any of the other channels of the set of channels. The first channel (222) may be configured without external fluid flow during the culture of the cell sample (230). For example, a first set of plugs (242) may be coupled to the ends of the first channel (222) to prohibit external fluid flow from an external source such as a fluid pump (250, 252) and fluid sources (260, 261, 262, 263). Fluid (270, 272) may flow into the first channel (222) through the first scaffold (220) over time.

Similar to the first cell culture device, a second cell culture device (202) may include a second substrate (211) defining a cavity in which a second scaffold (221) is disposed. The second substrate (211) and the second scaffold (221) may collectively define a set of channels (223, 225, 227) including a fourth channel (223) and a fifth channel (225). The fourth channel (223) may be configured to receive and culture a second cell sample (231) during use. In some embodiments, the fourth channel (223) may be configured to receive one or more of phosphate buffered saline and fetal bovine serum. The fifth channel (225) may be configured to receive the first fluid (270) and/or migrating cells from the first cell culture device (201) during use. The second scaffold (221) may be configured to permit diffusion of the fluid through the second scaffold (221) and into the fourth channel (223) from any of the other channels of the set of channels. The fourth channel (223) may be configured without external fluid flow during the culture of the second cell sample (231). For example, a second set of plugs (243) may be coupled to the ends of the fourth channel (223) to prohibit external fluid flow from an external source such as a fluid pump (250, 252) and fluid sources (260, 261, 262, 263). Fluid (270, 272) may flow into the fourth channel (223) through the second scaffold (221) over time. As shown in FIG. 2A, the first cell culture device (201) may be disposed in series with the second cell culture device (202).

A set of fluid pumps (250, 252) may be coupled to one or more channels of the set of channels (222, 223, 224, 225, 226, 227). The set of fluid pumps (250, 252) may include one or more peristaltic pumps. For example, a first fluid pump (250) may be coupled to the second channel (224) and the fifth channel (225) and a second fluid pump (252) may be coupled to the third channel (226) and the sixth channel (227). A set of fluid sources (260, 261, 262, 263) may be coupled to the set of fluid pumps (250, 252). For example, the first fluid pump (250) may be coupled to a first fluid source (260) and a second fluid source (261). The second fluid pump (252) may be coupled to a third fluid source (262) and a fourth fluid source (263).

Furthermore, a first connector (280) (e.g., tube) may couple the first fluid pump (250), the first fluid source (260), and the second fluid source (261) to the second channel (224) and the fifth channel (225). Likewise, a second connector (282) may couple the second fluid pump (252), the third fluid source (262), and the fourth fluid source (263) to the third channel (264) and the sixth channel (227).

In the configuration shown in FIG. 2A, fluid flows (270, 272) in the opposite direction through the fluidic channels (224, 226, 225, 227). However, it should be understood that the direction of fluid flow and location of the fluid pumps may be modified. In some embodiments, the set of fluid sources (260, 261, 262, 263) may be formed of the same substrate material as the cell culture devices (201, 202). The set of fluid sources (260, 261, 262, 263) may be configured to store fluid (e.g., cell growth media, therapeutic agent, pharmacological agent, drug, combinations thereof, and the like). The set of fluid pumps (250, 252) may be configured to recirculate the fluid (270, 272) through a closed circuit of the system (100).

As shown in the block diagram of FIG. 8A, a radiation source (810) may be configured to emit a light signal that illuminates the cell sample (230, 231) of either cell culture device (201, 202). A detector (814) may be configured to receive the light signal reflected from the cell sample (230, 231). A controller (822) of a control device (820) may be coupled to the detector (814) and include a processor and memory. In some embodiments, the controller (822) may be configured to receive signal data corresponding to the light signal received by the detector (814), generate cell sample data using the signal data, and identify one or more phenotypes of the cell sample (230, 231) using the cell sample data. In some embodiments, the phenotype may include at least one of non-invasive cells, invasive cells, size, shape, location, volume, growth rate, cell morphology, migration speed, DNA, RNA, and protein.

The set of channels (222, 223, 224, 225, 226) may be parallel. In some embodiments, the first channel (222) and the second channel (224) may have a center-to-center distance of between about 1 mm and about 5 mm. The set of channels (222, 223, 224, 225, 226) may include a third channel (226) having a center-to-center distance from the first channel (122) of between about 1 mm and about 5 mm. In some embodiments, the fourth channel (223) and the fifth channel (225) may have a center-to-center distance of between about 1 mm and about 5 mm. The set of channels (222, 223, 224, 225, 226) may include a sixth channel (227) having a center-to-center distance from the fourth channel (223) of between about 1 mm and about 5 mm.

In some embodiments, the third channel (226) and the sixth channel (227) may be configured to receive a fluid. In some of these embodiments, the fluid received in the second channel (224) and the fifth channel (225) may be a first fluid, and the fluid received in the third channel (226) and the sixth channel (227) may be a second fluid different from the first fluid. At least one of the first fluid and the second fluid may be a growth medium. The second channel (224), third channel (226), fifth channel (225), and sixth channel (226) may be independently configured to receive the fluid in a predetermined direction as described in more detail herein. In other embodiments, the third channel (226) and the sixth channel (227) may be configured without flow of a fluid. For example, at least one of the second channel (224), the third channel (226), fifth channel (225), and the sixth channel (227) may include a valve (not shown) that may control fluid flow including flow rate and direction.

In some embodiments, each channel of the set of channels (222, 223, 224, 225, 226) may be on a same plane. In other embodiments, at least one channel of the set of channels (222, 223, 224, 225, 226) may be on a different plane than the other channels of the set of channels. In some embodiments, each channel of the set of channels (222, 223, 224, 225, 226) may have a diameter of between about 0.2 mm and about 2.0 mm.

The set of channels (222, 223, 224, 225, 226) may include, respectively, a set of anchors (240, 241) that attach to an interface between the substrate (210, 211) and the scaffold (220, 221). For example, the fifth channel (225) and sixth channel (227) may each include a pair of anchors (240) spanning at least a portion of an interface between the second substrate (211) and the second scaffold (221). The substrates (210, 211) may each define a set of openings through which the set of anchors (240, 241) and plugs (242, 243) may be disposed. As described herein, the length of the anchors of the set of channels may differ to control interstitial pressure within different regions of the scaffold.

In some embodiments, the first channel (222) may be configured to receive the first cell sample (230) having a volume of at least about 20 μL. The first channel (222) may be configured to receive between about 10⁷ cells and about 10⁵ cells. The first cell sample may include one or more mammalian cells including cancer cells, epithelial cells, fibroblasts, immune cells, endothelial cells, and nerve cells. The cancer cells may include prostate cancer cells. In some embodiments, the third channel (223) may be configured to receive the second cell sample (231) having a volume of at least about 20 μL. The third channel (223) may be configured to receive between about 10⁷ cells and about 10⁵ cells. The second cell sample (231) may include one or more mammalian cells including tissue, cancer cells, epithelial cells, fibroblasts, immune cells, endothelial cells, and nerve cells. The cancer cells may include prostate cancer cells. The tissue of the second cell sample (231) may include tissue from organs that may commonly develop metastases.

In some embodiments, fluid may flow through the devices (201, 202) between about 1 day and about 28 days. In some of these embodiments, the fluid may be received continuously or substantially continuously. In some embodiments, a portion of the scaffold between the first channel (222) and the second channel (224) may have a pressure of between about 0 Pa and about 200 Pa during use. In some embodiments, a portion of the first scaffold (220) between the first channel (222) and the third channel (226) may have a pressure of between about 0 Pa and about 200 Pa during use. Similarly, in some embodiments, a portion of the second scaffold (221) between the fourth channel (223) and the fifth channel (225) may have a pressure of between about 0 Pa and about 200 Pa during use. In some embodiments, a portion of the second scaffold (221) between the fourth channel (223) and the sixth channel (227) may have a pressure of between about 0 Pa and about 200 Pa during use.

The first and second substrates (210, 211) may form sealed chambers. That is, the systems described herein may be sealed from an external environment. In some embodiments, the first and second substrates (210, 211) may include a height of between about 3 mm to about 20 mm, a length of between about 10 mm and about 30 mm, and a width of between about 1 mm and about 20 mm. The substrates (210, 211) may be composed of one or more of polysiloxane, polydimethylsiloxane, polystyrene, polyethylene, and polycarbonate. In some embodiments, the substrates (210, 211) may be composed of polydimethylsiloxane in a 11:1 polymer-to-cross-linker volume ratio. In some embodiments, one or more sides of the chamber may include a transparent portion (e.g., glass) that allows imaging of the cell culture in the scaffold (220, 221) to aid analysis of cell growth, invasion, and/or migration.

In some embodiments, the first and second scaffolds (220, 221) may be an extracellular matrix. The extracellular matrix may be composed of one or more of collagen, hydrogel, agarose, polyethylene glycol, alginate, hyaluronan acid, laminin, fibronectin, proteoglycans, and elastin. In some embodiments, the collagen may have a concentration of between about 1 mg/ml and about 10 mg/ml.

In some embodiments, the set of culture devices may include three, four, five, six, seven, or more culture devices arranged in series where some cell culture devices include cancer cells and other cell culture devices include mammalian cells such as organ tissue. In other embodiments, some of the culture devices may be disposed in parallel with additional or fewer fluid pumps and fluid sources, as necessary to generate and maintain a desired flow rate. In other embodiments, a set of culture devices may be configured in series and parallel. For example, a first set of culture devices arranged in series may be configured in parallel to a second set of culture devices arranged in series.

FIG. 2B is an illustrative block diagram of another cell culture system (250) having a set of cell culture devices (251, 252, 253). The system (250) may include a first cell culture device (251), a second cell culture device (252), and a third cell culture device (253) each coupled to a set of fluid pumps (250, 252) and a set of fluid sources (260, 261, 262, 263) (e.g., reservoirs). The cell culture devices (251, 252, 253), fluid sources (260, 261, 262, 263), and fluid pumps (250, 252) in FIG. 2B may be similar to the cell culture devices (e.g., 101, 201, 202), fluid sources (260, 261, 262, 263), and fluid pumps (250, 252) as described herein and are not repeated. That is, elements of the system (250) may share the same elements as those described with respect to the system (200). In FIG. 2B, the set of fluid pumps (250, 252) may be disposed between respective fluid sources (260, 261, 262, 263). A set of connectors (280, 282, 290) may fluidically couple the set of fluid pumps (250, 252), the set of fluid sources (260, 261, 262, 263), and the set of cell culture devices (251, 252, 253) to each other. The second cell culture device (252) and the third cell culture device (253) may be parallel to each other and may be commonly coupled to the first cell culture device (251).

In some embodiments, the first cell culture device (251) may include two fluidic channels. In some embodiments, the output of the first cell culture device (251) may be fluidically coupled to a third connector (290). The third connector (290) may mix the outputs of the two fluidic channels of the first cell culture device (251). In other embodiments, the second and third cell culture devices (252, 253) may each be coupled to one of the two fluidic channels. The third connector (290) may fluidically couple to one or more channels of the second and third cell culture devices (252, 253).

### Cell Culture Analysis System

Described herein are cell culture analysis systems that may include one or more of the components necessary to perform analysis of cell culture samples according to various embodiments described herein. For example, the analysis systems described herein may optically image, process, and analyze a cell sample to generate cell sample data. For example, the cell sample data may correspond to one or more phenotypes of the cell sample. Generally, the analysis systems described herein may include one or more of a radiation source (e.g., illumination source), a detector, and a controller (including memory, a processor, and computer instructions). The radiation source may be configured to emit a light signal (e.g., light beam) and to illuminate the cell sample. A detector may be configured to receive the light beam reflected by the cell sample. A controller coupled to the detector may be configured to receive signal data corresponding to the light beam received by the detector and generate cell sample data using the signal data. One or more phenotypes of the cell sample may be identified and characterized using the cell sample data. Any of the cell culture devices as described herein may be analyzed using the analysis systems as described herein.

### Radiation Source

The fluid analysis systems as described herein may include a radiation source configured to emit a first light signal (e.g., illumination) directed at the scaffold through, for example, a glass enclosure of the sealed chamber of the cell culture device. In some embodiments, the radiation source may include one or more of a light emitting diode, laser, microscope, optical sensor, lens, and flash lamp. For example, the radiation source may generate light that may be carried by fiber optic cables or one or more LEDs may be configured to provide illumination. In another example, a fiberscope including a bundle of flexible optical fibers may be configured to receive and propagate light from an external light source.

### Detector

Generally, the fluid analysis systems described herein may include a detector used to receive light signals (e.g., light beams) reflected by a cell sample in a cell culture device. The received light may be used to generate signal data that may be processed by a processor and memory to generate cell sample data. The detector may further be configured to image one or more identifiers (e.g., label, barcode) and identifiers of the cell culture device and/or cell sample. In some embodiments, the detector may include one or more of a lens, camera, and measurement optics. For example, the detector may include an optical sensor (e.g., a charged coupled device (CCD) or complementary metal-oxide semiconductor (CMOS) optical sensor) and may be configured to generate an image signal that is transmitted to a display. For example, the detector may include a camera with an image sensor (e.g., a CMOS or CCD array with or without a color filter array and associated processing circuitry).

### Control Device

The cell sample analysis systems as described herein may couple to one or more control devices (e.g., computer systems) and/or networks. FIG. 8B is a block diagram of the control device (820). The control device (820) may include a controller (822) including a processor (824) and a memory (826). In some embodiments, the control device (820) may further include a communication interface (830). The controller (822) may be coupled to the communication interface (830) to permit a user to remotely control the control device (820), radiation source (810), pump (812), detector (814), and any other component of the system (800). The communication interface (830) may include a network interface (832) configured to connect the control device (820) to another system (e.g., Internet, remote server, database) over a wired and/or wireless network. The communication interface (830) may further include a user interface (834) configured to permit a user to directly control the control device (820).

### Controller

Generally, the fluid analysis systems described herein may include at least one cell culture device and corresponding control device coupled to a radiation source and detector. In some embodiments, a detector may be configured to generate signal data. The signal data may be received by a controller and used to generate cell sample data corresponding to one or more phenotypes of a cell sample. The control device may accordingly identify and/or characterize one or more phenotypes of a cell sample. As described in more detail herein, the controller (822) may be coupled to one or more networks using a network interface (832). The controller (822) may include a processor (824) and memory (826) coupled to a communication interface (830) including a user interface (834). The controller (822) may automatically perform one or more steps of image processing and analysis, and thus improve one or more of specificity, sensitivity, and speed of cell sample analysis.

The controller (822) may include computer instructions for operation thereon to cause the processor (824) to perform one or more of the steps described herein. In some embodiments, the computer instructions may be configured to cause the processor to receive signal data from the detector, generate cell sample data using the signal data, and identify one or more characteristics of the cell sample using the analyte data. In some embodiments, the computer instructions may be configured to cause the controller to set imaging data parameters. The computer instructions may be configured to cause the controller to generate the cell sample data. Signal data and analysis may be saved for each channel of each cell culture device.

A control device (820), as depicted in FIG. 8B, may include a controller (822) in communication with the cell sample analysis system (800) (e.g., radiation source (810), pump (812), and detector (814)). The controller (822) may include one or more processors (824) and one or more machine-readable memories (826) in communication with the one or more processors (824). The processor (824) may incorporate data received from memory (826) and user input to control the system (800). The memory (826) may further store instructions to cause the processor (824) to execute modules, processes, and/or functions associated with the system (800). The controller (822) may be connected to and control one or more of a radiation source (810), pump (812), detector (814), communication interface (830), and the like by wired and/or wireless communication channels.

The controller (822) may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on a server or server computing devices such as routing/connectivity components, multiprocessor systems, microprocessor-based systems, distributed computing networks, personal computing devices, network appliances, portable (e.g., hand-held) or laptop devices. Examples of portable computing devices include smartphones, personal digital assistants (PDAs), cell phones, tablet PCs, wearable computers taking the form of smartwatches and the like, and portable or wearable augmented reality devices that interface with the patient's environment through sensors and may use head-mounted displays for visualization, eye gaze tracking, and user input.

### Processor

The processor (824) may be any suitable processing device configured to run and/or execute a set of instructions or code and may include one or more data processors, image processors, graphics processing units, physics processing units, digital signal processors, and/or central processing units. The processor (824) may be, for example, a general purpose processor, Field Programmable Gate Array (FPGA), an Application Specific Integrated Circuit (ASIC), combinations thereof, and the like. The processor (824) may be configured to run and/or execute application processes and/or other modules, processes and/or functions associated with the system and/or a network associated therewith. The underlying device technologies may be provided in a variety of component types including metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, and the like.

### Memory

In some embodiments, the memory (826) may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, combinations thereof, and the like. As used herein, database refers to a data storage resource. The memory (826) may store instructions to cause the processor (824) to execute modules, processes, and/or functions associated with the control device (820), such as calibration, indexing, signal processing, image analysis, cell sample analysis, notification, communication, authentication, user settings, combinations thereof, and the like. In some embodiments, storage may be network-based and accessible for one or more authorized users. Network-based storage may be referred to as remote data storage or cloud data storage. Signal data and analysis stored in cloud data storage (e.g., database) may be accessible to authorized users via a network, such as the Internet. In some embodiments, database (840) may be a cloud-based FPGA.

Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for a specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs); holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other embodiments described herein relate to a computer program product, which may include, for example, the instructions and/or computer code disclosed herein.

The systems, devices, and methods described herein may be performed by software (executed on hardware), hardware, or a combination thereof. Hardware modules may include, for example, a general-purpose processor (or microprocessor or microcontroller), a field programmable gate array (FPGA), an application specific integrated circuit (ASIC), combinations thereof, and the like. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

### Communication Interface

The communication interface (830) may permit a user to interact with and/or control the system (800) directly and/or remotely. For example, a user interface (834) of the system (800) may include an input device for a user to input commands and an output device for a user and/or other users (e.g., technicians) to receive output (e.g., view sample data on a display device) related to operation of the system (800). In some embodiments, a network interface (832) may permit the control device (820) to communicate with one or more of a network (870) (e.g., Internet), remote server (850), and database (840) as described in more detail herein.

### User Interface

User interface (834) may serve as a communication interface between a user (e.g., operator) and the control device (820). In some embodiments, the user interface (834) may include an input device and output device (e.g., touch screen and display) and be configured to receive input data and output data from one or more sensors, input device, output device, network (870), database (840), and server (850). For example, signal data generated by a detector may be processed by processor (824) and memory (826), and output visually by one or more output devices (e.g., display). Signal data, image data, and/or cell sample data may be received by user interface (834) and output visually, audibly, and/or through haptic feedback through one or more output devices. As another example, user control of an input device (e.g., joystick, keyboard, touch screen) may be received by user interface (834) and then processed by processor (824) and memory (826) for user interface (834) to output a control signal to one or more components of the fluid analysis system (800). In some embodiments, the user interface (834) may function as both an input and output device (e.g., a handheld controller configured to generate a control signal while also providing haptic feedback to a user).

### Output Device

An output device of a user interface (834) may output image data and/or analyte data corresponding to a sample and/or system (800), and may include one or more of a display device, audio device, and haptic device. The display device may be configured to display a graphical user interface (GUI). The user console (860) may include an integrated display and/or video output that may be connected to output to one or more generic displays, including remote displays accessible via the internet or network. The output data may also be encrypted to ensure privacy and all or portions of the output data may be saved to a server or database. A display device may permit a user to view signal data, calibration data, tissue data, image data, cell sample data, system data, fluid data, patient data, and/or other data processed by the controller (822). In some embodiments, an output device may include a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, holographic display, combinations thereof, and the like.

An audio device may audibly output patient data, image data, cell sample data, system data, alarms and/or warnings. For example, the audio device may output an audible warning upon malfunction of a fluid pump. In some embodiments, an audio device may include at least one of a speaker, piezoelectric audio device, magnetostrictive speaker, and/or digital speaker. In some embodiments, a user may communicate with other users using the audio device and a communication channel.

A haptic device may be incorporated into one or more of the input and output devices to provide additional sensory output (e.g., force feedback) to the user. For example, a haptic device may generate a tactile response (e.g., vibration) to confirm user input to an input device (e.g., joystick, keyboard, touch surface). In some embodiments, the haptic device may include a vibrational motor configured to provide haptic tactile feedback to a user. Additionally or alternatively, haptic feedback may notify a user of an error such as pump malfunction and/or tube disconnection. This may prevent potential harm to the system.

### Input Device

Some embodiments of an input device may include at least one switch configured to generate a control signal. For example, the input device may be configured to control one or more pumps to control fluid flow rate. In some embodiments, the input device may include a wired and/or wireless transmitter configured to transmit a control signal to a wired and/or wireless receiver of a controller (822). For example, an input device may include a touch surface for a user to provide input (e.g., finger contact to the touch surface) corresponding to a control signal. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In embodiments of an input device including at least one switch, a switch may include, for example, at least one of a button (e.g., hard key, soft key), touch surface, keyboard, analog stick (e.g., joystick), directional pad, pointing device (e.g., mouse), trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio and recognize a user voice as a control signal.

### Network Interface

As depicted in FIG. 8A, a control device (820) described herein may communicate with one or more networks (870) and computer systems (850) through a network interface (832). In some embodiments, the control device (820) may be in communication with other devices via one or more wired and/or wireless networks. The network interface (832) may facilitate communication with other devices over one or more external ports (e.g., Universal Serial Bus (USB), multi-pin connector) configured to couple directly to other devices or indirectly over a network (e.g., the Internet, wireless LAN).

In some embodiments, the network interface (832) may include a radiofrequency receiver, transmitter, and/or optical (e.g., infrared) receiver and transmitter configured to communicate with one or more devices and/or networks. The network interface (832) may communicate by wires and/or wirelessly with one or more of the sensors, user interface (834), network (870), database (840), and server (850).

In some embodiments, the network interface (832) may include radiofrequency (RF) circuitry (e.g., RF transceiver) including one or more of a receiver, transmitter, and/or optical (e.g., infrared) receiver and transmitter configured to communicate with one or more devices and/or networks. RF circuitry may receive and transmit RF signals (e.g., electromagnetic signals). The RF circuitry converts electrical signals to/from electromagnetic signals and communicates with communications networks and other communications devices via the electromagnetic signals. The RF circuitry may include one or more of an antenna system, an RF transceiver, one or more amplifiers, a tuner, one or more oscillators, a digital signal processor, a CODEC chipset, a subscriber identity module (SIM) card, memory, and the like. A wireless network may refer to any type of digital network that is not connected by cables of any kind.

Examples of wireless communication in a wireless network include, but are not limited to cellular, radio, satellite, and microwave communication. The wireless communication may use any of a plurality of communications standards, protocols and technologies, including but not limited to Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, near-field communication (NFC), radio-frequency identification (RFID), Wireless Fidelity (Wi-Fi) (e.g., IEEE 802.11a, IEEE 802.11b, IEEE 802.11g, IEEE 802.11n), Voice over Internet Protocol (VoIP), Wi-MAX, a protocol for email (e.g., Internet Message Access Protocol (IMAP), Post Office Protocol (POP)), instant messaging (e.g., eXtensible Messaging and Presence Protocol (XMPP), Session Initiation Protocol for Instant Messaging, Presence Leveraging Extensions (SIMPLE), Instant Messaging and Presence Service (IMPS)), Short Message Service (SMS), or any other suitable communication protocol. Some wireless network deployments combine networks from multiple cellular networks or use a mix of cellular, Wi-Fi, and satellite communication.

In some embodiments, a wireless network may connect to a wired network in order to interface with the Internet, other carrier voice and data networks, business networks, and personal networks. A wired network is typically carried over copper twisted pair, coaxial cable, and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, wireless personal area networks (PAN) (e.g., Bluetooth, Bluetooth Low Energy), and virtual private networks (VPN). As used herein, network refers to any combination of wireless, wired, public, and private data networks that are typically interconnected through the Internet, to provide a unified networking and information access system.

### III. Methods

Described herein are embodiments corresponding to methods of using a cell culture device for analyzing a cell sample such as mammalian cells and/or cancer cells and manufacturing a cell culture device. These methods may identify and/or characterize a cell sample and in some embodiments, may be used with the systems and devices described. For example, a cell sample analysis system may culture a cell sample placed in a scaffold and identify one or more phenotypes of the cell sample.

### Culturing a Cell Sample

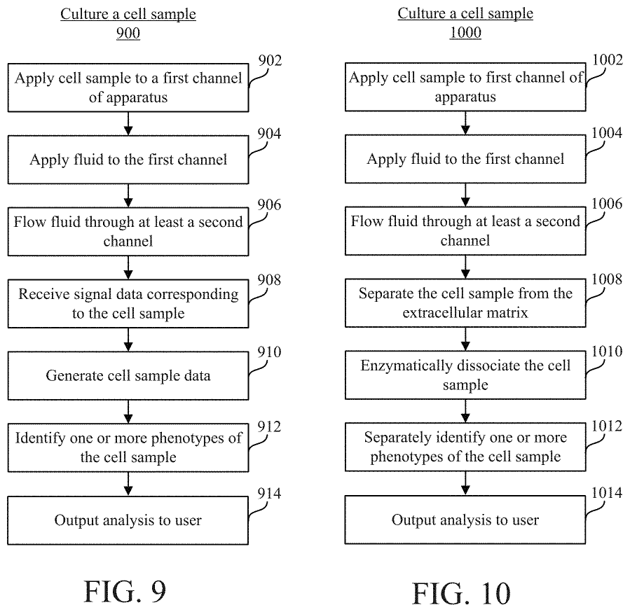

Methods for culturing a cell sample in some embodiments may use a cell sample analysis system and/or cell culture device as described herein. The methods described herein may quickly and easily identify phenotypes from a cell sample based on optical analysis techniques and/or physical separation. FIG. 9 is a flowchart that generally described a method of culturing a cell sample (900) that may be optically analyzed. A cell culture device structurally and/or functionally similar to the cell culture devices as described herein may used in one or more of the steps described herein. The process may include step 902 of applying a cell sample to a first channel of a cell culture device. For example, a needle and syringe containing a fluidic suspension of mammalian cells may be used to introduce the cell sample into the device. The cell sample may have a volume of at least about 20 μL. The cell sample may include one or more of mammalian cells and cancer cells including prostate cancer cells. The first channel may be configured to receive between about 10⁷ cells and about 10⁵ cells. In some embodiments, substantially the entire first channel may be filled with the cell sample and fluid suspension. At step 904, a fluid may be applied to the first channel. For example, one or more of phosphate buffered saline and fetal bovine serum may be applied to the first channel. For example, a syringe may deliver 1% v/v fetal bovine serum (FBS) inserted into the first channel through the openings in the substrate and anchor lumen. After removal of the syringe, a set of plugs may be used to close the ends of the first channel. A set of vents may be formed in the scaffold that allow fluid (e.g., liquid, air) to escape when the cell sample and/or fluid is applied to the first channel. For example, the set of vents may be coupled to the first channel.

In some embodiments, the device may include a substrate defining a cavity and a scaffold disposed within the cavity. The substrate and the scaffold may collectively define a set of channels including the first channel and a second channel parallel to the first channel. The first channel and the second channel may have a center-to-center distance of between about 1 mm and about 5 mm. The set of channels may include a third channel having a center-to-center distance from the first channel of between about 1 mm and about 5 mm. Each channel of the set of channels may have a diameter of between about 0.2 mm and about 2.0 mm. In some embodiments, each channel of the set of channels may be on a same plane. In other embodiments, at least one channel of the set of channels may be on a different plane than the other channels of the set of channels. In some embodiments, the substrate may be composed of one or more of polysiloxane, polydimethylsiloxane, polystyrene, polyethylene, and polycarbonate. The substrate may be composed of polydimethylsiloxane in a 11:1 polymer-to-cross-linker volume ratio. In some embodiments, the scaffold may be an extracellular matrix. The extracellular matrix may be composed of one or more of collagen, hydrogel, agarose, polyethylene glycol, alginate, hyaluronan acid, laminin, fibronectin, proteoglycans, and elastin. The collagen may include a concentration between about 1 mg/ml and about 10 mg/ml. The scaffold may be enclosed and sealed from an external environment.

In some embodiments, prior to applying the cell sample to the first channel, a set of anchors may be attached to an interface between the substrate and the scaffold to secure the scaffold to the substrate. Attaching the set of anchors may include a first anchor attached to the first channel and a second anchor attached to the second channel.

At step 906, fluid may flow through at least the second channel using a fluid pump for a set of predetermined time periods (e.g., for at least about 21 days). External fluid flow may be prohibited through the first channel after applying the cell sample to the first channel such that the first channel is subject to indirect interstitial pressure from the flowing fluid through the second channel. The fluid flowing through the second channel and/or the third channel may flow at a rate of between about 1 μL/min and about 200 μL/min. In some embodiments, fluid may flow through the second channel and/or the third channel for the set of predetermined time periods. In some embodiments, the fluid flowing through the second channel and/or the third channel may flow at a rate of between about 1 μL/min and about 200 μL/min. The fluid may flow substantially continuously. The set of predetermined time periods may collectively be between about 1 day and about 60 days. The fluid flowing in the second channel may be a first fluid, and the fluid flowing in the third channel may be a second fluid. At least one of the first fluid and the second fluid may be a growth medium. Air bubbles may be prohibited from forming through the second channel using, for example, a fluid source as described herein. Fluid may flow through the second channel in a closed loop path.

In some embodiments, the flowing fluid through the second channel and the third channel may be in a same direction or an opposite direction. In some embodiments, external fluid flow through the first channel and/or third channel may be prohibited after applying the cell sample to the first channel. A portion of the scaffold between the first channel and the second channel may have a pressure of between about 0 Pa and about 200 Pa during the fluid flow. A portion of the scaffold between the first channel and the third channel may have a pressure of between about 0 Pa and about 200 Pa during the fluid flow.

At step 908, signal data corresponding to the cell sample may be received at a set of predetermined time intervals. In some embodiments, the time intervals may be between about 1 minute and 1 day. At step 910, cell sample data may be generated from the detector using a processor and memory as described herein. At step 912, one or more phenotypes of the cell sample may be identified from the cell sample data. For example, cancer cells derived from patient biopsies or surgical resections may be cultured in the device and tested for the phenotypic ability to invade as a diagnostic measure of clinical outcome or disease aggressiveness. Signal data such as the speed of invasion and the number of cells that invade may be calculated, analyzed, and utilized as diagnostic biomarkers. Patients with a slow or small number of invading cells may be classified as less aggressive disease whereas patients with a faster or larger number of invading cells may be classified as more aggressive disease. Moreover, if the patient is undergoing treatment with a certain drug or therapeutic agent, the cultured cells derived from patient may be subjected to same treatment as the patient to determine for any cell subpopulations that may confer a resistant phenotype to the cancer cells. Signal data such as the ability of the cancer cells to form a mass in the first channel and the ability to invade while under exposure to treatment may be used as an output to predict if the patient will experience disease progression while under therapy or develop resistance to therapy. At step 914, the analysis performed may be output by the cell sample analysis system. In some embodiments, machine learning algorithms may be used to accelerate and automate analysis of the signal data generated for diagnostic or prognostic readouts.

### Analyzing a Cell Sample

FIG. 10 is a flowchart that generally described a method of culturing a cell sample (100) that may be physically dissociated for analysis. This may allow the cultured cell sample to physically separate an invasive cell sample from a non-invasive cell sample. A cell culture device structurally and/or functionally similar to the cell culture devices as described herein may used in one or more of the steps described herein. The process may include step 1002 of applying a cell sample to a first channel of a cell culture device. Elements and substeps of steps 1002, 1004, and 1006 may share the same elements and substeps of steps 902, 904, and 906 and are not repeated.

At step 1008, a portion of the cell sample may be separated from the extracellular matrix. For example, the cell sample may be cut out of the extracellular matrix. At step 1010, the cell sample may be enzymatically dissociated into an invasive portion and a non-invasive portion, enabling the isolation of live cells from each portion. The cell sample may be first extracted by peeling off the top transparent layer from the culture device. Using a low-power objective stereomicroscope, a pair of micro-scissors (e.g., Fine Science Tools) and forceps (e.g., Fine Science Tools) may be used to remove portions of the collagen around the non-invasive portion that contains the invasive portion which can be visually demarcated. The non-invasive portion bearing non-invasive cells may be physically extracted into a separate environment and subjected to enzymatic dissociation using about 0.25% w/v collagenase A (commercially available from Sigma-Aldrich and reconstituted in about a 0.25% w/v ratio in a neutral buffered solution such as 1× phosphate buffered saline, pH 7.4) at about 37° C. to release any live invasive cells not removed by excision. The excised collagen bearing the invasive portion may also subjected to enzymatic dissociation using about 0.25% w/v collagenase A to isolate the invasive cells from the collagen into a single live cell suspension. During enzymatic dissociation, sufficient collagenase may be added to enclose the about 0.1% collagen gel. For enzymatic dissociation of the non-invasive portion, the process may periodically visualized under the microscope (e.g., about every 2 minutes) to ensure sufficient release of any surrounding invasive cells without disrupting the integrity of the non-invasive portion. To dissociate the non-invasive portion into live single cell suspension, the non-invasive portion may be further treated with about 0.25% w/v trypsin solution for about 5 minutes at about 37° C. Collagenase A, trypsin, and the concentrations described are merely illustrative and not limited. At step 1012, one or more phenotypes of one or more of the invasive portion and the non-invasive portion may be separately identified. Identification of the molecular phenotypes of the invasive and non-invasive portion may be carried out on single or bulk cells through a variety of molecular approaches including one or a combination of DNA or RNA microarray, DNA or RNA sequencing or proteomic approaches such as mass-spectrometry-based approaches, reverse phase protein array or immunoblotting. At step 1014, the analysis performed may be output by the cell sample analysis system.

### Manufacturing a Device

Also described herein are embodiments corresponding to methods for manufacturing a cell culture device and fluid source that may be used in some embodiments with the cell culture system embodiments as described herein. Any of the devices (101, 201, 211, 251, 252, 253, 300, 302, 304, 306, 308, 400, 500, 600, etc.) as described herein may manufactured using one or more of the manufacturing steps described herein. For example, the methods described here may manufacture a cell culture system using injection molding techniques.

Generally, the methods described herein include forming a substrate and an extracellular matrix disposed within a cavity therein. A substantially transparent layer may be coupled to the substrate to enclose and seal the extracellular matrix from an external environment. For example, the substrate and extracellular matrix may be formed by polymerization.

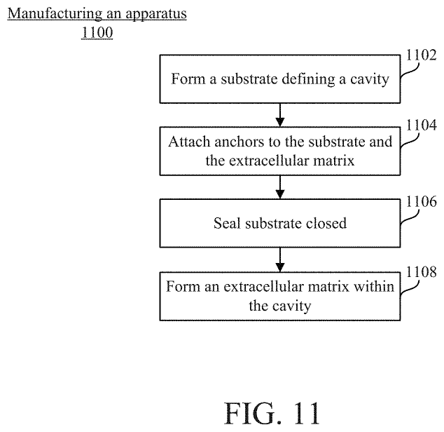

FIG. 11 is a flowchart that generally describes a method of manufacturing a cell culture device (1100). The process may include step 1102 of forming a substrate defining a cavity and step 1104 of forming an extracellular matrix within the cavity. For example, uncured polydimethylsiloxane may be poured into a mold and cured over a predetermined set of time at around 70° C. to form the substrate with a predetermined set of channels, openings, and/or vents.

The substrate and the extracellular matrix may collectively define a set of channels including a first channel configured to three-dimensionally culture a cell sample and a second channel parallel to the first channel. The first channel and the second channel may have a center-to-center distance of between about 1 mm and about 5 mm.

In some embodiments, forming the substrate at step 1102 may include the substeps of polymerizing polysiloxane to form a predetermined shape. For example, as shown in FIG. 5, the predetermined shape may include a cavity (550) and a set of vents (560) defined in the substrate (510). In some embodiments, the polysiloxane may be disposed over a set of parallel rods (540). The rods (540) may be removed from the substrate (510) such that the substrate defines a portion of the set of channels. In some embodiments, the substrate may be composed of one or more of polysiloxane, polydimethylsiloxane, polystyrene, polyethylene, and polycarbonate. The substrate may be composed of polydimethylsiloxane in about an 11:1 polymer-to-cross-linker volume ratio.

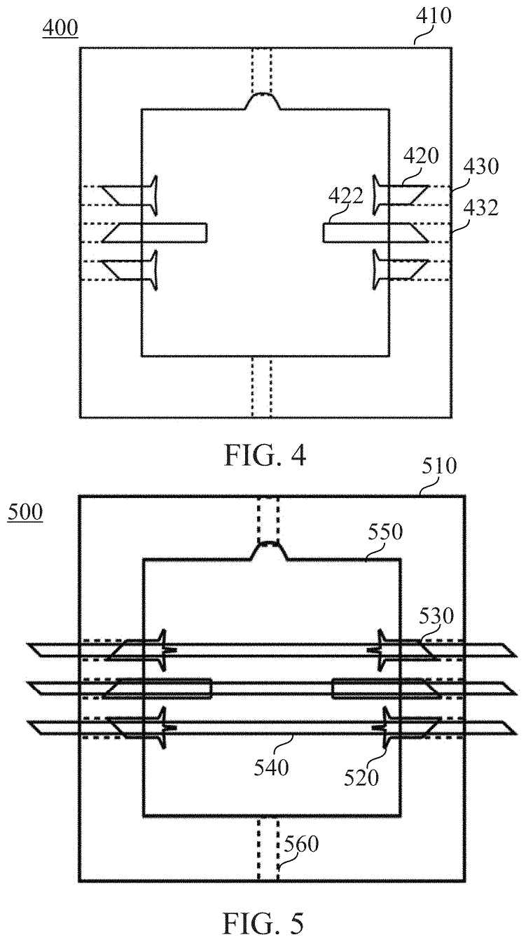

At step 1104, a set of anchors may be attached to an interface between the substrate and the extracellular matrix to be formed in the cavity. As shown in FIG. 4A, for example, the set of anchors may include a first anchor (420) attached to the first channel (430) and a second anchor (422) attached to the second channel (432). At step 1106, the substrate (410) may be sealed closed. For example, a substantially transparent layer may be coupled to the substrate. In some embodiments, the substrate and the substantially transparent layer may be coupled using one or more of adhesives, ultrasonic welding, laser welding, and solvent bonding. In some embodiments, the substrate and the substantially transparent layer may be formed using one or more of die cutting, extrusion, additive manufacturing, stereolithography, fused deposit modeling, and injection molding.

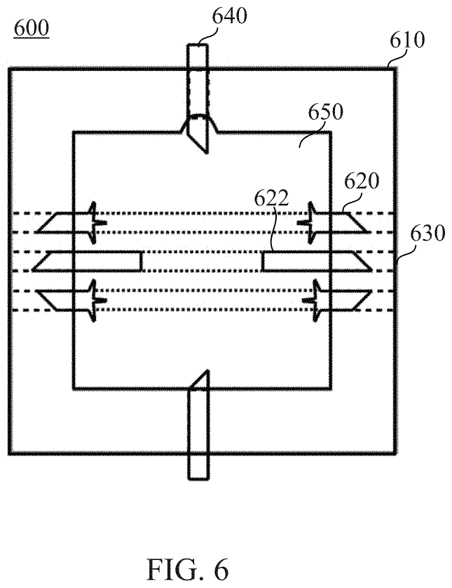

In some embodiments, forming the extracellular matrix at step 1108 may include the substeps of inserting the set of parallel rods through the extracellular matrix and polymerizing the extracellular matrix disposed over the set of parallel rods within the cavity. For example, the extracellular matrix may polymerize for about 30 minutes at about 37° C. As shown in FIG. 6, the set of parallel rods may be removed from the substrate (610) and the extracellular matrix (650) such that the substrate (610) and the extracellular matrix (650) defines the set of channels (630). In some embodiments, the extracellular matrix may be composed of one or more of collagen, hydrogel, agarose, polyethylene glycol, alginate, hyaluronan acid, laminin, fibronectin, proteoglycans, and elastin. The collagen may includes a concentration between about 1 mg/ml and about 10 mg/ml. In some embodiments, 0.1% collagen I may be used. In some embodiments, air may escape through a set of openings in the substrate when the cavity is filled with the extracellular matrix. A set of plugs (640) may close one or more openings in the substrate (610). A set of anchors (620) may be disposed in the set of channels (630).

Methods of manufacturing a fluid source (700) may include similar steps to forming the substrate of the cell culture device described herein. For example, uncured polydimethylsiloxane may be poured into a mold and cured over a predetermined set of time at around 70° C. to form a fluid source substrate (710) with a predetermined set of openings (704, 706) and a cavity (702). In some embodiments, a cavity (702) of the fluid source substrate (710) may be formed by using a core punch to form a circular hollow interior as shown in FIG. 7A. In some embodiments, the substrate (710) may be composed of one or more of polysiloxane, polydimethylsiloxane, polystyrene, polyethylene, and polycarbonate. The substrate (710) may be composed of polydimethylsiloxane in about an 11:1 polymer-to-cross-linker volume ratio. In some embodiments, the fluid source cavity (700) may have a diameter of between about 10 mm and about 30 mm. An inlet (704) and outlet (706) of the fluid source (700) may have a diameter of between about 1 mm and about 10 mm.

In some embodiments, one or more transparent layers (720, 722) may be coupled to opposite sides of the substrate (710) to allow imaging of the fluid and/or any cells within the fluid source. For example, a first transparent layer (720) may be composed of PDMS having a thickness of about 0.5 mm and a second transparent layer (722) may be composed of glass having a thickness of about 1.0 mm. The first and second transparent layers (720, 722) may be coupled to the fluid source substrate (710) using a plasma oxidizer cleaner. High pressure steam may be used to sterilize the devices and fluid sources described herein.

### IV. Examples

As described herein, the cell culture devices as described herein may generate a variety of mechanical and fluid dynamic forces such as interstitial flow and during long-term cell culture (e.g., weeks, months) in a sealed three-dimensional environment.

A. Fluid Flow and Interstitial Pressure

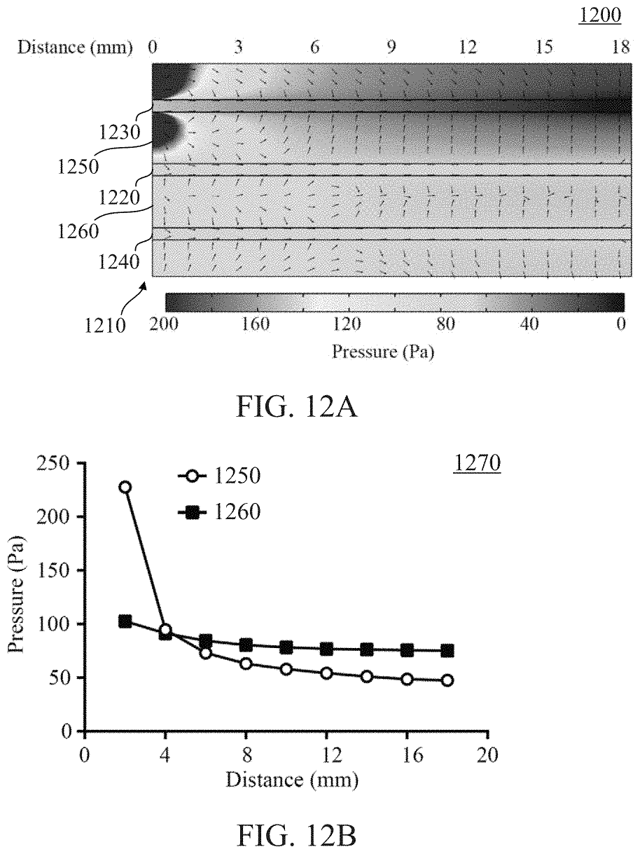

FIG. 12A is a fluid flow vector and interstitial fluidic pressure diagram of a cell culture device (1200). In particular, FIG. 12A depicts a plan view of a scaffold (1210) of a cell culture device (1200). The scaffold (1210) defines a first channel (1220), second channel (1230), and third channel (1240). In FIG. 12A, the first channel (1220) and the third channel (1240) do not receive fluid flow while the second channel (1230) is subject to fluid flow at a rate of about 30 μl/min. The portions of the scaffold (1210) between the channels are herein referred to as interstitial space having an interstitial pressure. The scaffold (1210) may include a first interstitial space (1250) and a second interstitial space (1260) between respective channels. The set of arrows within the scaffold (1210) correspond to the fluid flow vector (e.g., interstitial flow) throughout the cell culture device (1200). FIG. 12B is a graph (1270) of the interstitial pressure relative to distance of the cell culture device (1200) depicted in FIG. 12A for the first interstitial space (1250) and the second interstitial space (1260).

B. Cell Culture Growth Over Time

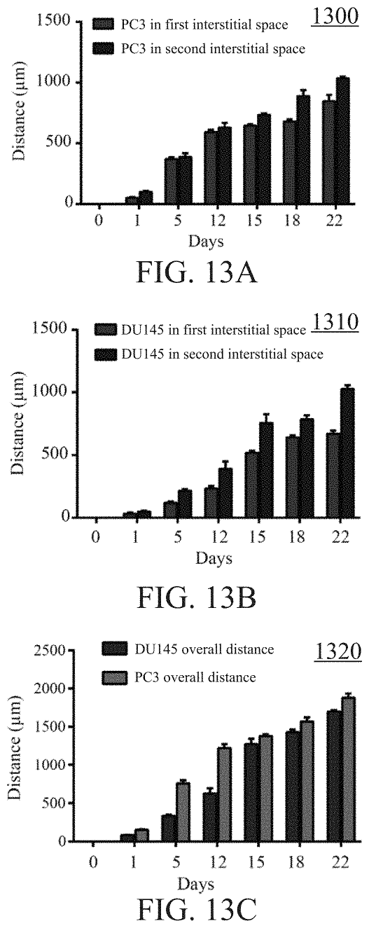

Separate cell culture devices were seeded in a first channel with respective prostate cancer cells DU145 and PC3. A growth medium fluid was continuously circulated through a second channel of the cell culture devices at a rate of about 30 μl/min for about 21 days. A third channel was maintained without fluid flow as a control. FIGS. 13A-13C are illustrative graphs (1300, 1310, 1320) depicting cell growth of the cell samples DU145 and PC3 over time. Invasion was measured on both sides of the first channel. The graph (1300) of FIG. 13A illustrates a migration distance of PC3 cells in different regions of the cell culture device over time. The graph (1310) of FIG. 13B illustrates a migration distance of DU145 cells in different regions of the cell culture device over time. The graph (1320) of FIG. 13C illustrates an overall migration distance of PC3 cells relative to DU145 cells over time. As shown in FIGS. 13A-13C, the measured invasion rates vary in different regions of interstitial space as well as between cell lines. In the first two weeks of culture, PC3 cells were more invasive than DU145 cells based on a maximum invasion distance (defined as the longest distance from a leader cell to the first channel), which is consistent with the known invasive potential difference between the two cell lines. However, after three weeks of culture, this difference was reduced except in the first interstitial space.

For both DU145 and PC3 tumoroids, the distance of cell invasion into the scaffold (e.g., collagen gel) was greater in the second interstitial space than in the first interstitial space at three weeks. This difference in cancer cell invasion distance between different regions of interstitial space may be due to an increased fluid pressure within the second interstitial space, which is known to affect tumor invasion. This suggests that interstitial pressure and flow is an important factor in cell invasion. While PC3 cells were overall two times more invasive than DU145 cells after twelve days of cell culture, this difference was rapidly lost by 22 days, suggesting that the invasive potential of cells may change over time, thereby highlighting the importance of long-term tumoroid culture as provided by the devices, systems, and methods as described herein.

C. Interstitial Pressure and Extracellular Matrix Concentration

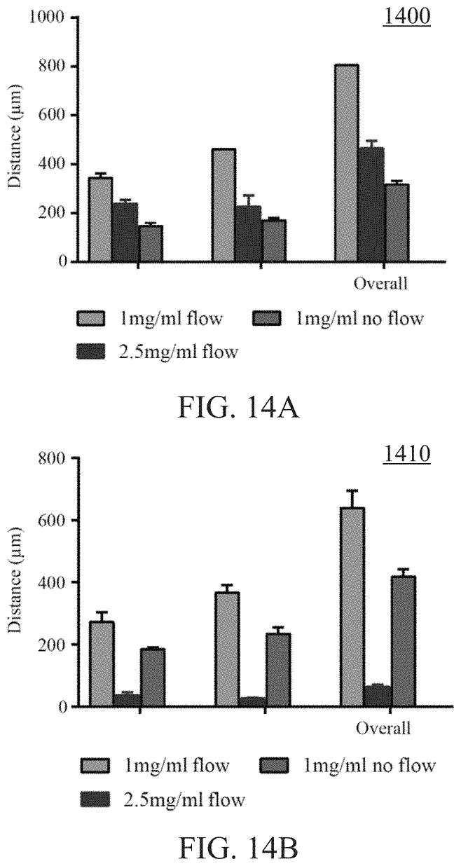

FIGS. 14A-14B are illustrative graphs depicting cell growth (e.g., invasion distance) as a function of fluid flow rate and extracellular matrix (e.g., collagen) concentration. FIG. 14A illustrates a migration distance of PC3 cells as a function of location, flow rate, and concentration. FIG. 14B illustrates a migration distance of DU145 cells as a function of location, flow rate, and concentration. For PC3 cells, increasing collagen concentration from 1 mg/ml to 2.5 mg/ml without affecting flow reduced the maximum invasion in both the first and second interstitial spaces by about two-fold (FIG. 14A). However, a similar increase in collagen concentration in DU145 tumoroids resulted in a greater decrease in maximum invasion in both the first and second interstitial spaces regions by about ten-fold (FIG. 14B).

Furthermore, tumoroids were grown in 1 mg/ml and 2.5 mg/ml of collagen in a no flow condition where growth medium was added manually daily. For PC3 tumoroids, the exclusion of flow in cell culture devices with 1 mg/ml of collagen reduced maximum invasion by about 2.5-fold. For DU145 tumoroids in cell culture devices with 1 mg/ml of collagen, maximum invasion was reduced by about 1.5-fold. The exclusion of flow in devices with 2.5 mg/ml collagen resulted in no observable invasion by about one week for either PC3 or DU145 tumoroids.

D. Phenotype Analysis of Cells—Staining

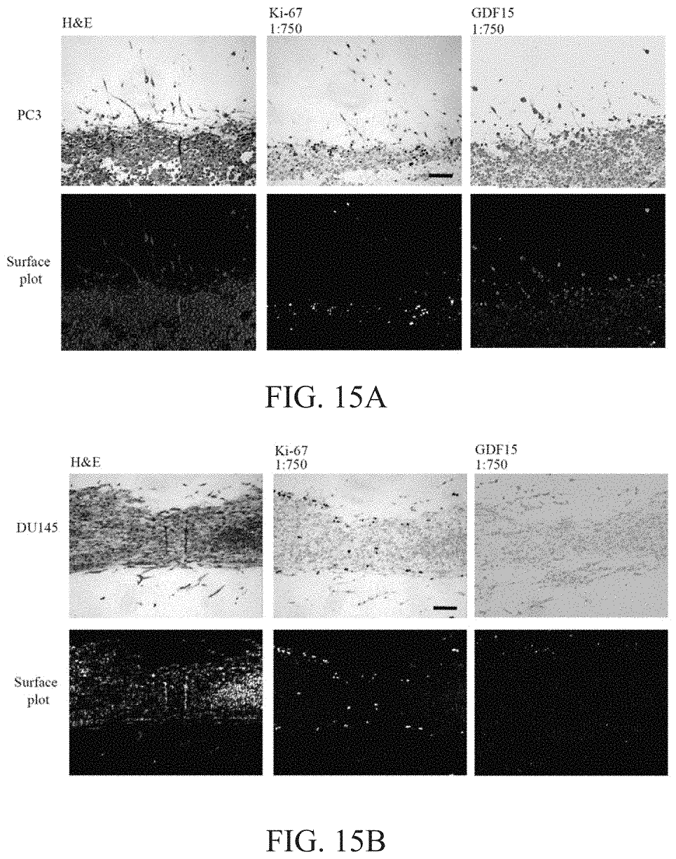

FIG. 15A includes images comparing phenotypes for PC3 cells that migrated and other PC3 cells that were non-invading, and stained using hematoxylin and eosin (H&E), Ki-67, and GDF15. FIG. 15B includes images comparing phenotypes for DU145 cells that migrated and other DU145 cells that were non-invading, and stained using hematoxylin and eosin (H&E), Ki-67, and GDF15. Phenotypes of the invading cells were compared to phenotypes of the non-invading cells. In particular, histological sections of the extracted scaffold bearing mammalian cells were stained with hematoxylin and eosin (H&E) and for Ki-67. Ki-67 is a molecular marker of cell proliferation and metastasis. H&E staining revealed in situ features such as DU145 cells arranged in layers of cells near the interface between the tumoroid and scaffold. By contrast, the PC3 tumoroid was arranged in clusters of round cells at the interface. Furthermore, prominent Ki-67 staining was mainly localized at the interface between the tumoroid and the scaffold or in invading cells in both PC3 and DU145 tumoroids. Tumoroids were also stained for growth differentiation factor 15 (GDF15), a marker found to be elevated in the serum levels of metastatic prostate cancer patients and which may be strongly associated with poor clinical outcomes. Both PC3 and DU145 tumoroids demonstrated greater GDF15 expression among the invading cells and along the interface between the tumoroid and scaffold. A quantitative heat map of the immunohistochemical signal intensity (“Surface plot”) is provided in FIGS. 15A and 15B below each of the stains.

E. Phenotype Analysis of Cells—Extraction

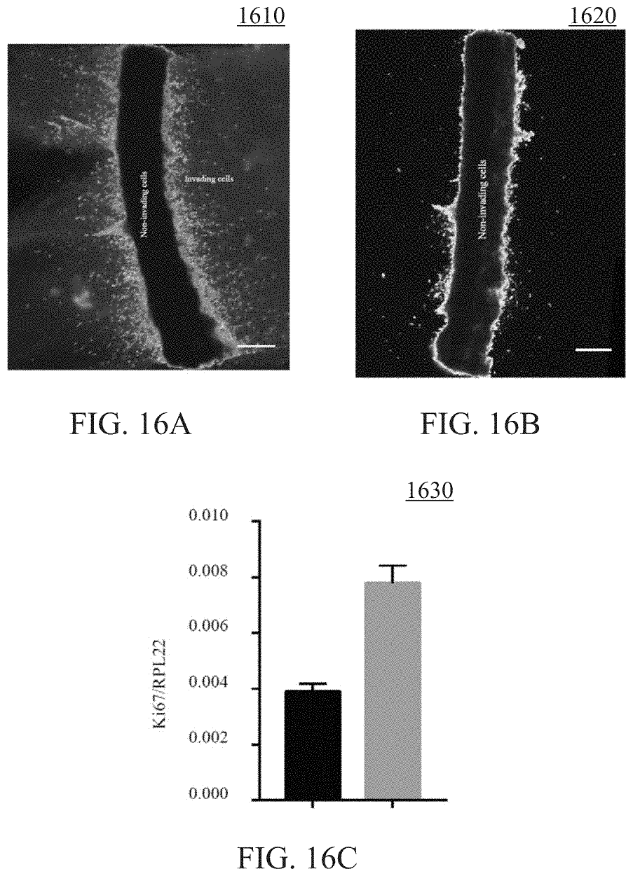

Subpopulations of cancer cells may be separated based on invasive potential to isolate and allow molecular characterization of the cell subsets. FIG. 16A is an image of an extracted tumoroid having invasive and non-invasive cells after cell culture in a cell culture device. In FIG. 16A, invasive and non-invasive cells were embedded in 0.1% collagen and cultured for 21 days. FIG. 16B is an image of the extracted tumoroid depicted in FIG. 16A after enzymatic dissociation. FIG. 16C is a graph of real-time polymerase chain reaction (q-RT-PCR) analysis of target gene Ki-67 expression relative to an RPL22 reference gene of the sample imaged in FIG. 16A for an invading cell subpopulation and a non-invading cell subpopulation.

The devices, systems, and methods of use thereof may provide long-term culture of subpopulations of cells (such as invasive or non-invasive) and sufficient spatial resolution between the subpopulations. This may further allow separation of these two subpopulations using macro dissection and enzymatic dissociation techniques for isolation and analysis.

The invasive cells were observed visually under a light microscope as cells invading outward from the tumoroid indicated as the dark mass in the center which bears the non-invading cells. The tumoroid was extracted by opening the sealed chamber (e.g., peeling off a PDMS layer of the substrate) of the cell culture device. Next, the substrate (e.g., 0.1% collagen gel) was trimmed using a pair of micro-scissors and forceps under a light microscope at low power 2× objective to remove portions of the collagen that did not bear any cells. The resulting trimmed 0.1% collagen gel bearing invasive and non-invasive cell subpopulations was physically extracted from the device using the forceps and subjected to enzymatic dissociation using 0.25% w/v collagenase A (commercially available from Sigma-Aldrich and reconstituted in a 0.25% w/v ratio in a neutral buffered solution such as 1× phosphate buffered saline, pH 7.4) at 37° C. During enzymatic dissociation, sufficient collagenase was added to enclose the 0.1% collagen gel. The dissociation process was visualized at predetermined time intervals under the microscope (e.g., every two minutes). This was done to ensure that the time period for enzymatic dissociation was sufficient to release the invasive cells from the 0.1% collagen gel without disrupting the integrity of the tumoroid bearing the non-invading subpopulation.

In FIG. 16B, single invading cells were no longer localized to the proximity of the non-invading tumoroid. Instead, the invading cells were in a suspension of 0.25% collagenase A. Once this degree of dissociation had occurred, the tumoroid bearing non-invasive cells was physically removed using forceps from the 0.25% collagenase A solution bearing the invasive subpopulation and processed separately.

The graph of FIG. 16C indicates that the Ki-67 expression levels of the invading cell subpopulation are higher than non-invading cell subpopulations by at least two-fold. This suggests that the two different subpopulations possess a different molecular phenotype.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

The foregoing description, for purposes of explanation, used specific nomenclature to provide a thorough understanding of various inventions and embodiments disclosed herein. However, it will be apparent to one skilled in the art that specific details are not required in order to practice the disclosed inventions and embodiments. Thus, the foregoing descriptions of specific embodiments of the inventions and corresponding embodiments thereof are presented for purposes of illustration and description. They are not intended to be exhaustive or to limit the invention to the precise forms disclosed; obviously, many modifications and embodiments are possible in view of the above teachings. The embodiments were chosen and described in order to best explain the principles of the inventions, the corresponding embodiments thereof, and practical applications, so as to enable others skilled in the art to best utilize the invention and various implementations with various modifications as are suited to the particular use contemplated. It is intended that the following claims and their equivalents define the scope of the invention.

In addition, any combination of two or more such features, structure, systems, articles, materials, kits, steps and/or methods, disclosed herein, if such features, structure, systems, articles, materials, kits, steps and/or methods are not mutually inconsistent, is included within the inventive scope of the present disclosure. Moreover, some embodiments of the various inventions disclosed herein may be distinguishable from the prior art for specifically lacking one or more features/elements/functionality found in a reference or combination of references (i.e., claims directed to such embodiments may include negative limitations).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms. This work was supported by an award from the American Heart Association.

1. A method, comprising:
applying a cell sample to a first channel of an apparatus, the apparatus including a substrate defining a cavity and further including a scaffold disposed within the cavity, wherein the substrate and the scaffold collectively define a set of channels including the first channel and a second channel parallel to the first channel;
prohibiting external fluid flow through the first channel after applying the cell sample to the first channel; and
flowing fluid through the second channel using a fluid pump for at least about 21 days such that the first channel is subject to indirect interstitial pressure from the fluid flowing through the second channel.

2. The method of claim 1, further comprising:
receiving signal data corresponding to the cell sample at a set of predetermined time intervals;
generating cell sample data from the detector; and
identifying one or more phenotypes of the cell sample from the cell sample data.

3. (canceled)

4. The method of claim 1, the flowing the fluid including flowing the fluid through the second channel in a closed loop path.

5-8. (canceled)

9. The method of claim 1, wherein the scaffold is enclosed and sealed from the external environment.

10-35. (canceled)

36. The method of claim 1, wherein the fluid flows continuously.

37-38. (canceled)

39. The method of claim 1, wherein the cell sample is a first cell sample, the apparatus is a first apparatus, the substrate is a first substrate, the cavity is a first cavity, the scaffold is a first scaffold, and the set of channels is a first set of channels; and
applying a second cell sample to a first channel of a second apparatus, the second apparatus including a second substrate defining a second cavity and a second scaffold disposed within the second cavity, wherein the second substrate and the second scaffold collectively define a second set of channels including the first channel and a second channel parallel to the first channel;
prohibiting external fluid flow through the second channel after applying the cell sample to the first channel
flowing the fluid through the second channel using the fluid pump for at least about 21 days such that the first channel is subject to indirect interstitial pressure from the flowing fluid through the second channel.

40. The method of claim 39, wherein the cell sample is a first cell sample; and
receiving signal data corresponding to the second cell sample at a set of predetermined time intervals;
generating second cell sample data from the detector; and
identifying one or more phenotypes of the second cell sample from the second cell sample data.

41. A method, comprising:
applying a cell sample to a first channel of an apparatus, the apparatus including a substrate defining a cavity and an extracellular matrix disposed within the cavity, wherein the substrate and the extracellular matrix collectively define a set of channels including a first channel configured to three-dimensionally culture a cell sample and a second channel parallel to the first channel;
prohibiting external fluid flow through the first channel after applying the cell sample to the first channel;
flowing fluid through the second channel using a fluid pump for at least about 21 days such that the first channel is subject to indirect interstitial pressure from the flowing fluid through the second channel;
separating a portion of the cell sample from the extracellular matrix;
enzymatically dissociating the cell sample into an invasive portion and a non-invasive portion;
identifying one or more phenotypes of one or more of the invasive portion; and
identifying one or more phenotypes of one or more of the non-invasive portion.

42. The method of claim 41, wherein each phenotype of the one or more phenotypes is selected from the group consisting of cell morphology, migration speed, DNA, RNA, and protein.

43-77. (canceled)

78. The method of claim 41, wherein the cell sample is a first cell sample, the apparatus is a first apparatus, the substrate is a first substrate, the cavity is a first cavity, the scaffold is a first scaffold, and the set of channels is a first set of channels; and
applying a second cell sample to a first channel of a second apparatus, the second apparatus including a second substrate defining a second cavity and a second scaffold disposed within the second cavity, wherein the second substrate and the second scaffold collectively define a second set of channels including the first channel and a second channel parallel to the first channel;
prohibiting external fluid flow through the second channel after applying the cell sample to the first channel; and
flowing the fluid through the second channel using the fluid pump for at least about 21 days such that the first channel is subject to indirect interstitial pressure from the flowing fluid through the second channel.

79. (canceled)

80. A method of manufacturing an apparatus, comprising:
forming a substrate defining a cavity;
forming an extracellular matrix within the cavity, wherein the substrate and the extracellular matrix collectively define a set of channels including a first channel configured to three-dimensionally culture a cell sample and a second channel parallel to the first channel; and
coupling a substantially transparent layer to the substrate to enclose and seal the extracellular matrix from an external environment.

81. The method of claim 80, wherein forming the substrate comprises:
polymerizing polydimethyl siloxane disposed over a set of parallel rods;
removing the rods from the substrate such that the substrate defines a portion of the set of channels; and
forming the cavity in the substrate.

82-84. (canceled)

85. The method of claim 80, wherein the first channel and the second channel have a center-to-center distance of between about 1 mm and about 5 mm.

86. The method of claim 80, further comprising attaching a set of anchors to an interface between the substrate and the extracellular matrix.

87-93. (canceled)

94. The method of claim 80, wherein each channel of the set of channels has a diameter of between about 0.2 mm and about 2.0 mm.

95-100. (canceled)

101. A system, comprising:
a first apparatus including a first substrate defining a first cavity and a first scaffold disposed within the first cavity, wherein the first substrate and the first scaffold collectively define a first set of channels including a first channel and a second channel, the first channel configured to receive and culture a first cell sample during use, the second channel configured to receive a fluid during use, the first scaffold configured to permit diffusion of the fluid through the first scaffold and into the first channel;
a second apparatus including a second substrate defining a second cavity and a second scaffold disposed within the second cavity, wherein the second substrate and the second scaffold collectively define a second set of channels including a third channel and a fourth channel, the third channel configured to receive and culture a second cell sample during use, the fourth channel configured to receive the fluid during use from the first apparatus, the second scaffold configured to permit diffusion of the fluid through the second scaffold and into the third channel;
a set of fluid pumps coupled to one or more of the first set of channels and the second set of channels; and
a set of fluid sources coupled to the set of fluid pumps, each fluid source coupled between a corresponding fluid pump and a corresponding channel of the first set of channels or the second set of channels, the first channel and the third channel configured to prohibit directly receiving fluid flow from the fluid pumped by the set of fluid pumps.

102. The system of claim 101, further comprising:
a radiation source configured to emit a light signal that illuminates one or more of the first cell sample and the second cell sample;
a detector configured to receive the light signal reflected from one or more of the first cell sample and the second cell sample; and
a controller coupled to the detector and including a processor and memory, wherein the controller is configured to:
receive signal data corresponding to the light signal received by the detector;
generate cell sample data using the signal data;
identify one or more phenotypes of the one or more first cell sample and the second cell sample using the cell sample data.

103. The system of claim 101, wherein the phenotype includes at least one of non-invasive cells, invasive cells, size, shape, location, volume, growth rate, cell morphology, migration speed, DNA, RNA, and protein.

104-135. (canceled)

136. The system of claim 101, wherein the fluid flows through the second channel and the fourth channel in a closed loop path.

137. The system of claim 101, further comprising a third apparatus fluidically coupled in series with the first apparatus.

138-167. (canceled)
